(19) United States
(12) Patent Application Publication
(10) Pub. No.: US 2018/0086782 A1
(43) Pub. Date: Mar. 29, 2018

(54) Guanidinoglycoside-mediated liposome-based delivery of therapeutics

(71) Applicant: The Regents of the University of California, Oakland, CA (US)

(72) Inventors: Jeffrey D. Esko, San Diego, CA (US); Yitzhak Tor, San Diego, CA (US)

(21) Appl. No.: 15/563,102

(22) PCT Filed: Apr. 1, 2016
(86) PCT No.: PCT/US16/25730

§ 371 Date: Sep. 29, 2017

Related U.S. Application Data
(60) Provisional application No. 62/141,769, filed on Apr. 1, 2015.

(51) Int. Cl. C07H 15/232; A61K 9/127

(52) U.S. Cl. CPC C07H 15/232; A61K 9/127

(57) ABSTRACT

This disclosure relates to the incorporation of amphiphilic guanidinylated aminoglycosides (e.g., neomycin) into liposomal assemblies, which contain entrapped therapeutics. The lysosome is responsible for enzymatically breaking down and recycling large biomolecules and aged organelles. While malfunctioned lysosomal enzymes have been established in Lysosomal Storage Disorders (LSDs), recent reports have suggested that defects in lysosomal enzymes (e.g., glucocerebrosidase) are also linked to other chronic ailments, including neurological disorders such as Parkinson's Disease and related disorders.

### CROSS-REFERENCE TO RELATED APPLICATIONS

This application claims priority to U.S. Provisional Application Ser. No. 62/141,769, filed Apr. 1, 2015, the disclosure of which is incorporated herein by reference in its entirety.

### FEDERALLY SPONSORED RESEARCH OR DEVELOPMENT

This invention was made with Government support under Grant No. GM077471 awarded by the National Institutes of Health and Grant No. CHE-1303554 awarded by the National Science Foundation. The Government has certain rights in the invention.

### TECHNICAL FIELD

This disclosure relates to the incorporation of amphiphilic guanidinylated aminoglycosides (e.g., neomycin) into liposomal assemblies, which contain entrapped therapeutics.

### BACKGROUND

The lysosome is responsible for enzymatically breaking down and recycling large biomolecules and aged organelles.¹ While malfunctioned lysosomal enzymes have been established in Lysosomal Storage Disorders (LSDs),² recent reports have suggested that defects in lysosomal enzymes (e.g., glucocerebrosidase) are also linked to other chronic ailments, including neurological disorders such as Parkinson's Disease and related disorders.³⁻⁵

### SUMMARY

The present application provides, inter alia, a compound of Formula I:

A-B-C I

or a pharmaceutically acceptable salt thereof, wherein:

A is a guanidinylated neomycin derivative;

B is a linker group; and

C is a phospholipid, a fatty acid, or a fatty acid group.

In some embodiments, A is a guanidinylated neomycin derivative of the following formula:

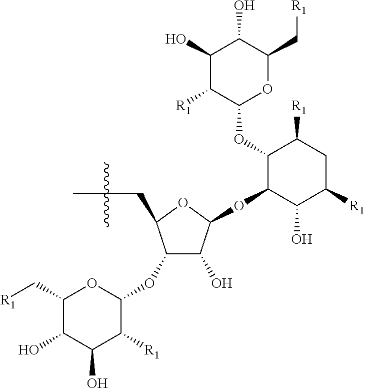

wherein:

R₁ is a guanidine or guanidinium group; and

[symbol] indicates the bond between A and B of Formula I.

In some embodiments, the guanidine group is an N-protected guanidine. In some embodiments, the N-protected guanidine group is of the following formula:

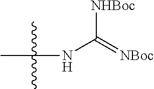

wherein Boc is tert-butoxycarbonyl.

In some embodiments, the guanidinium group is of the following formula:

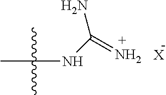

wherein X⁻ is an anion.

In some embodiments, the guanidinium group is of the following formula:

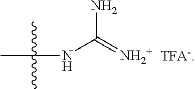 [SMILES: *NC(N)=[NH2+]]

In some embodiments, B is a linker group comprising a linker selected from the group consisting of one or more alkylene groups, one or more amide groups, one or more alkyleneoxy groups, one or more heteroaryl groups, one or more amine groups, or any combination thereof. In some embodiments, B is a linker group comprising one or more C₁₋₁₀ alkylene groups, one or more —(OCH₂CH₂)— or —(OCH₂)— groups, one amide group, one —NH— group, and one 5-6 membered heteroaryl group. In some embodiments, B is a linker group selected from the group of the following formulae:

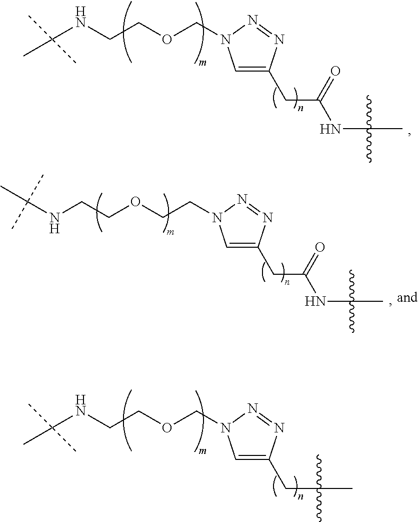

wherein:

m is an integer from 1 to 10;

n is an integer from 0 to 10;

[symbol] indicates the bond between A and B of Formula I; and

[symbol] indicates the bond between B and C of Formula I.

In some embodiments, m is 3.

In some embodiments, n is 0 or 3.

In some embodiments, m is an integer from 1 to 5. In some embodiments, m is 3. In some embodiments, n is an integer from 0 to 5. In some embodiments, n is 0 or 3.

In some embodiments, C is a phospholipid. In some embodiments, C is a phospholipid comprising one or more choline groups, one or more glycerophosphoric acid groups, and one or more fatty acid groups. In some embodiments, C is a phospholipid comprising one or more ethanolamine groups, one or more glycerophosphoric acid groups, and one or more fatty acid groups.

In some embodiments, C is a phosphatidylcholine. In some embodiments, C is a phosphatidylcholine selected from the group consisting of POPC (1-palmitoyl-2-oleoyl-sn-glycero-3-phosphocholine), DOPC (1,2-dioleoyl-sn-glycero-3-phosphocholine), DOPE (1,2-dioleoyl-sn-glycero-3-phosphoethanolamine), or any combination thereof.

In some embodiments, C is a phospholipid, a fatty acid, or a fatty acid group selected from the following formulae:

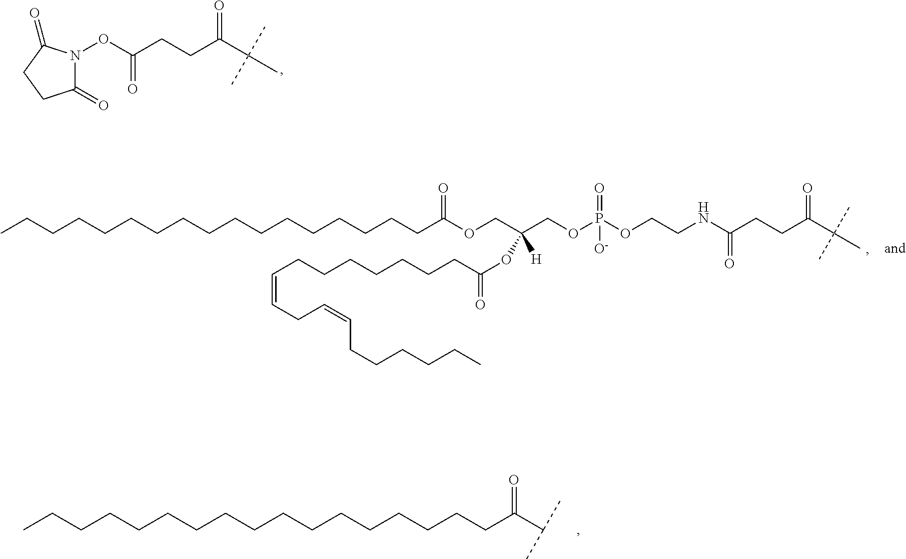

wherein [symbol] indicates the bond between B and C of Formula I.

In some embodiments, the compound of Formula I is selected from the group consisting of:

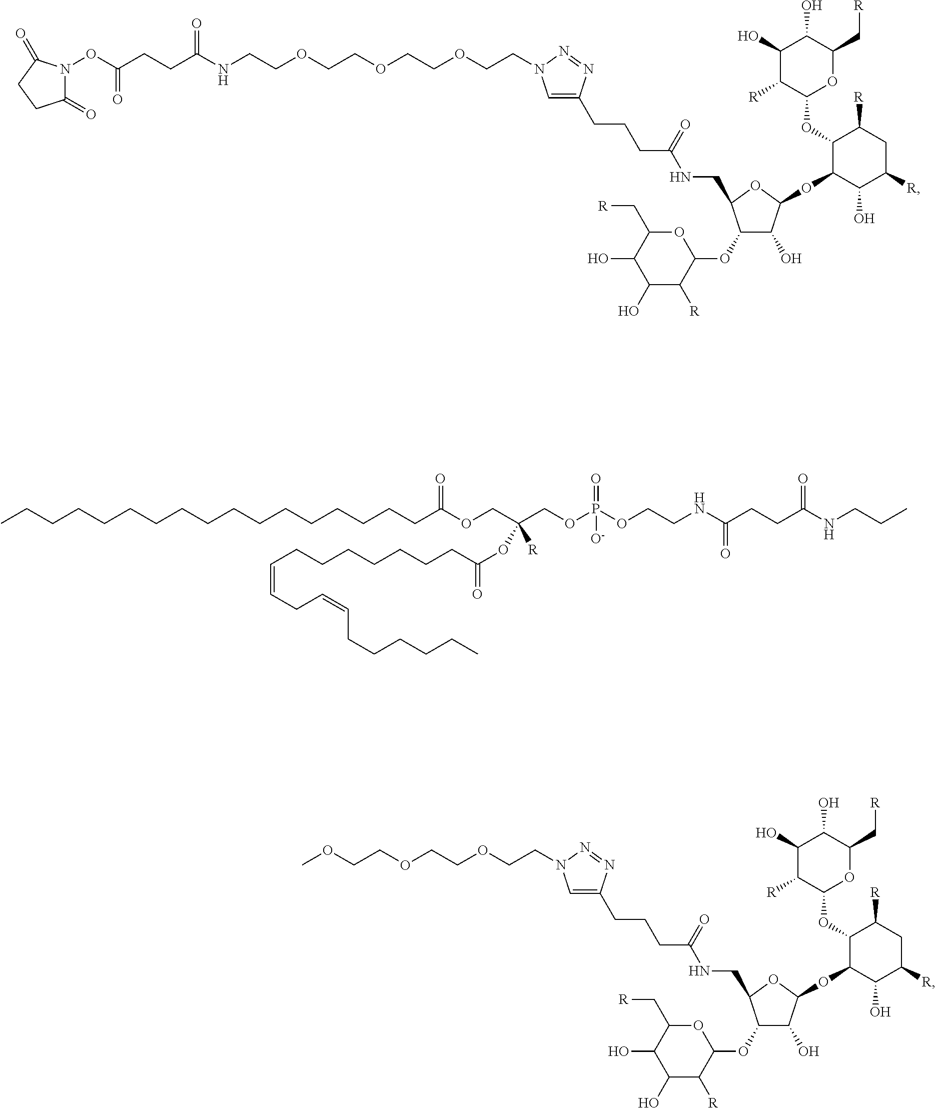

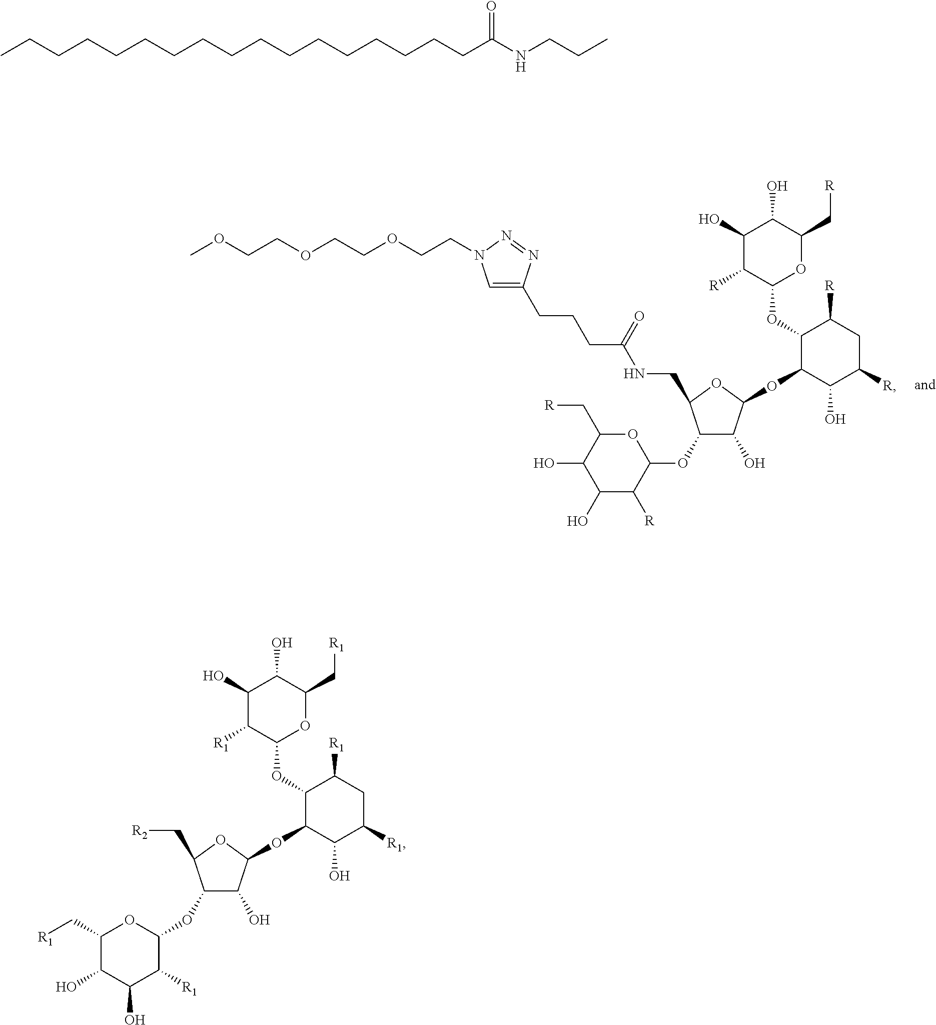

wherein:

each R is

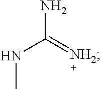

each R₁ is

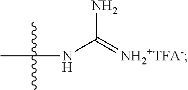

and

each R₂ is

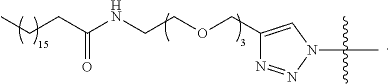

The present application further provides a conjugate, comprising a compound provided herein and a liposome.

In some embodiments, the liposome comprises a group selected from the group consisting of a POPC group (1-palmitoyl-2-oleoyl-sn-glycero-3-phosphocholine) derivative, a DOPC group (1,2-dioleoyl-sn-glycero-3-phosphocholine), DOPE (1,2-dioleoyl-sn-glycero-3-phosphoethanolamine), and a cholesterol group, or any combination thereof.

In some embodiments, the conjugate is selected from the group consisting of POPC:Stearyl-GNeo, DOPC:Stearyl-GNeo, DOPC:DOPE:Stearyl-GNeo, DOPC:DOPE:Cholesterol:Stearyl-GNeo. In some embodiments, the ratio of POPC:Stearyl-GNeo is about 100:1. In some embodiments, the ratio of DOPC:Stearyl-GNeo is about 100:0.9. In some embodiments, the ratio of DOPC:DOPE:Stearyl-GNeo it about 85:15:0.9. In some embodiments, the ratio of DOPC:DOPE:Cholesterol:Stearyl-GNeo is about 73:11:16:0.9.

In some embodiments, a conjugate provided herein further comprises a therapeutic agent.

The present application further provides a method for treating a Lysosomal Storage Disorder, a central nervous system (CNS) disorder or neurological disorder in a patient in need thereof, comprising administering to the patient a conjugate provided herein.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-1B shows plots of the size distribution (DLS) of GNeosomes (i.e., guanidinylated neomycin liposomes) (FIG. 1A) and plain liposomes (FIG. 1B).

FIG. 2 shows a plot of encapsulation efficiency (%). The lipid formulations are as follows: 1) POPC; 2) DOPC; 3) DOPC:DOPE 85:15; 4) DOPC:DOPE:Cholesterol 73:11:16; 5) POPC:Stearyl-GNeo 100:1; 6) DOPC:Stearyl-GNeo, 100:0.9; 7) DOPC:DOPE:Stearyl-GNeo 85:15:0.9; 8) DOPC:DOPE:Cholesterol:Stearyl-GNeo 73:11:16:0.9; 9) DOPC:DOPE:Cholesterol:DOTAP 73:11:16:0.9; 10) DOPC:DOPE:Cholesterol:DOTAP 73:11:16:5.4.

FIG. 3A shows a plot of cellular uptake of plain liposomes (right bars) and GNeo-decorated liposomes (left bars). Cells were incubated with lipid vesicles for 1 h at 37° C. Mean fluorescence intensity (MFI) was measured by flow cytometry. The background signal from untreated cells was subtracted. Wild type CHO-K1 cells incubated with plain and GNeo-decorated liposomes prepared with the indicated lipid composition. PC-PE-Ch=DOPC:DOPE:cholesterol, 73:11:16; PC-PE=DOPC:DOPE, 85:15. All GNeo-decorated liposomes contained a 0.9% mol stearyl-GNeo (i.e., Compound 4).

FIG. 3B shows a plot of cellular uptake of plain liposomes (right bars) and GNeo-decorated liposomes (left bars). HEK293T and Hep3B cells were incubated with the indicated lipid vesicles (0.3 mg mL⁻¹) for 1 h at 37° C. Mean fluorescence intensity (MFI) was measured by flow cytometry. The background signal from untreated cells was subtracted.

FIG. 4A shows a plot of cellular viability of cells incubated with plain liposomes (left bars) and GNeosomes (right bars). CHO-K1 cells were incubated for 24 hours with plain liposomes or GNeosomes at the indicated concentrations in serum-free medium. Medium was replaced and Cell titer blue was added. Cell viability was calculated by measuring the fluorescence intensity at 530/580.

FIG. 4B shows a plot of mean fluorescence intensity (MFI) of cells incubated with GNeosomes. CHO-K1 cells and mutant pgsA-745 cells incubated with GNeosomes at the indicated concentrations.

FIG. 5A-5F show flow cytometry data of the cellular delivery of Cyanine derivative (Cy5). CHO-K1 and psg-A745 cells were incubated with GNeosomes and plain liposomes loaded with Cy5 for one hour at 37° C. and subsequently analyzed by FACS. Upper panels (FIGS. 5A-5C): GNeosomes. Lower panels (FIGS. 5D-5F): Plain liposomes. Untreated CHO-K1 cells, treated psg-A745 cells, and treated CHO-K1 cells are shown. Liposome concentrations: FIGS. 5A and 5D: 100 μm/mL; FIGS. 5B and 5E: 300 μm/mL; FIGS. 5C and 5F: 500 μm/mL.

FIG. 6A shows Z-potential of the evaluated liposomes. G=GNeosomes, N=DOTAP-N, M=DOTAP-M and P=plain liposomes.

FIG. 6B shows mean fluorescence intensity (MFI) of CHO-K1 cells incubated with GNeosomes, lipid vesicles modified with DOTAP and plain liposomes, all consisting of PC-PE-Choi 73:11:16 at 300 DOTAP-M contains 0.9% mol DOTAP and DOTAP-N contains 5.4% mol DOTAP.

FIG. 7A shows normalized mean fluorescence intensity (MFI) of CHO-K1 cells incubated with GNeosomes. CHO-K1 cells were incubated with GNeosomes (300 mL⁻¹) at 37° C. and at 4° C. Cells were treated with amiloride (Am, 10 minutes, 5 μM) or sucrose (Suc, 1 hour, 400 mM) at 37° C. prior to incubation with GNeosomes. The background signal from untreated cells was subtracted and the MFI was normalized.

FIG. 7B shows normalized mean fluorescence intensity (MFI) of CHO-K1 cells incubated with plain liposomes. CHO-K1 cells were incubated with plain liposomes (300 mL⁻¹) at 37° C. and at 4° C. Cells were treated with amiloride (Am, 10 minutes, 5 μM) or sucrose (Suc, 1 hour, 400 mM) at 37° C. prior to incubation with liposomes. The background signal from untreated cells was subtracted and the MFI was normalized.

FIGS. 8A-8F show intracellular localization of lipid vesicles. Upper panels (FIGS. 8A-8C): GNeosomes. Lower panels (FIGS. 8D-8F): Plain liposomes. FIGS. 8A and 8D: LysoTracker Green DND-26. FIGS. 8B and 8E: Vesicles loaded with Cy5; FIGS. 8C and 8F: merged images with nuclear Hoechst dye.

FIGS. 9A-9C show cellular uptake of GNeosomes loaded with Streptavidin-Cy3. FIG. 9A: LysoTracker Green DND-26; FIG. 9B: GNeosomes; FIG. 9C: merged images with Hoechst dye.

FIG. 10 shows flow cytometry of the cellular delivery of Streptavidin-Cy3. CHO-K1 cells were incubated with liposomes (at the indicated concentrations) loaded with Streptavidin-Cy3 for one hour at 37° C. and subsequently analyzed by FACS as described herein.

FIGS. 11A-11D show the release of cargo in the lysosomes. Upper panels (FIGS. 11A and 11C): GNeosomes loaded with LysoSensor™. Lower panels (FIGS. 11B and 11D): non-encapsulated LysoSensor™. FIGS. 11A-11B show merged images showing the “green” channel and LysoTracker Deep Red. FIGS. 11C-11D show ratiometric images showing the ratio between the fluorescent intensity of the “green” and “blue” channels.

FIGS. 12A-12H show CLSM images used to assess the lysosomal release of encapsulated cargo. CHO-K1 cells were incubated with GNeosomes loaded with LysoSensor™ Dextran Blue/Yellow or with unencapsulated LysoSensor™ Dextran Blue/Yellow. Upper panels (FIGS. 12A-12D): unencapsulated LysoSensor™. Lower Panels (FIGS. 12E-12H): GNeosomes loaded with LysoSensor™. FIGS. 12A and 12E: Lysotracker® Deep Red; FIGS. 12B and 12F: green channel; FIGS. 12C and 12G: blue channel; FIG. 12D shows a merged image of FIGS. 12A-12C; and FIG. 12H shows a merged image of FIGS. 12E-12G.

FIG. 13A shows flow cytometry of lysosomal targeting by liposomes loaded with FDG. CHO-K1 cells were incubated for one hour with GNeosomes or plain liposomes at the indicated concentrations. The background signal from untreated cells was subtracted and the ratio between the signals from GNeosomes and plain liposomes was calculated.

FIGS. 13B-13D show CLSM images of CHO-K1 cells incubated for one hour with GNeosomes loaded with FDG. FIG. 13B: Fluorescein, released from FDG; FIG. 13C: LysoTracker Deep Red; and FIG. 13D: an overlay of FIGS. 13B and 13C including the nuclear stain Hoechst.

FIG. 14 shows a comparison of three methodologies for preparing lipidated GNeosome derivatives.

FIG. 15 shows results of delivering α-L-Iduronidase (IDUA) into MPS cells taken from patients using “plain” liposomes and lipidated GNeosome derivatives.

### DETAILED DESCRIPTION

While cargo delivery to the nucleus, cytoplasm and mitochondria has been extensively addressed,^{6,7} lysosomal delivery has been mainly addressed by covalently linking cargo to carriers, only moderately enhancing lysosomotropism.⁸⁻¹² Efficient and specific delivery of intact unmodified cargo to the lysosome remains, however, a challenge despite its significant potential as a research tool and as a future therapeutic approach for lysosome-associated diseases.¹³

Over the past decade, a new family of non-toxic cellular delivery vehicles based on guanidinoglycosides have been developed.^{14,15} This family of synthetic carriers is made by converting all ammonium groups on aminoglycoside antibiotics to guanidinium groups.¹⁵ Unlike other guanidinium-rich transporters and cell penetrating peptides (e.g., Tat and oligoarginines), the cellular uptake of guanidinoglycosides occurs at nanomolar concentrations and exclusively depends on cell surface heparan sulfate (HS) proteoglycans.¹⁶ These highly charged cell surface biopolymeric receptors, which decorate all mammalian cells, thus provide a privileged high capacity pathway for entry into the cell.^{17,18} Studies suggest that high MW cargo, when conjugated to guanidinoglycosides, enter the cell complexed to heparan sulfate and localize in lysosomes, where glycosaminoglycans are stored and metabolized.¹⁹ Moreover, recent cell-surface FRET analysis suggests that the multivalent nature of these conjugates is directly responsible for proteoglycan aggregation, which appears to be a pivotal step for the endocytic translocation of these carriers and their ultimate lysosomal localization.^{20,21}

One feature of the above mentioned approach is the need to covalently link the intended cargo to the carrier, which presents the following predicaments. Conjugation of low MW bioactive agents to guanidinoglycosides might drastically alter their basic physical properties and certain proteins might not retain their full activity when modified in this manner. Encapsulating any cargo in a molecular container such as a liposome that presents guanidinoglycosides on its periphery, thus likely retaining the cell surface HS selectivity and entry pathways, could potentially circumvent this limitation and provide a universal lysosomal delivery vehicle for any cell impermeable cargo.

Provided herein are GNeosomes, lysosome-targeting nanocarriers. Fabricated by introducing amphiphilic guanidinylated neomycin (GNeo) into regular liposomes, these assemblies are capable of specifically delivering a wide variety of unmodified cargo (e.g., a therapeutic agent) into the lysosomes.

### Definitions

For the terms “for example” and “such as” and grammatical equivalences thereof, the phrase “and without limitation” is understood to follow unless explicitly stated otherwise. As used herein, the term “about” is meant to account for variations due to experimental error. All measurements reported herein are understood to be modified by the term “about”, whether or not the term is explicitly used, unless explicitly stated otherwise. As used herein, the singular forms “a,” “an,” and “the” include plural referents unless the context clearly dictates otherwise.

A “therapeutically effective amount” of a conjugate and/or compound (e.g., a therapeutic agent) with respect to the subject method of treatment, refers to an amount of the conjugate(s) and/or compound in a preparation which, when administered as part of a desired dosage regimen (to a patient, e.g., a human) alleviates a symptom, ameliorates a condition, or slows the onset of disease conditions according to clinically acceptable standards for the disorder or condition to be treated or the cosmetic purpose, e.g., at a reasonable benefit/risk ratio applicable to any medical treatment.

As used herein, the term “treating” or “treatment” includes reversing, reducing, or arresting the symptoms, clinical signs, and underlying pathology of a condition in manner to improve or stabilize a patient's condition. For example, the term “treating” or “treatment” refers to one or more of (1) inhibiting the disease; for example, inhibiting a disease, condition or disorder in an individual who is experiencing or displaying the pathology or symptomatology of the disease, condition or disorder (i.e., arresting further development of the pathology and/or symptomatology); and (2) ameliorating the disease; for example, ameliorating a disease, condition or disorder in an individual who is experiencing or displaying the pathology or symptomatology of the disease, condition or disorder (i.e., reversing the pathology and/or symptomatology) such as decreasing the severity of disease or reducing or alleviating one or more symptoms of the disease.

As used herein the term “GNeo”, used alone or in combination with other terms, refers to a “guanidinylated neomycin” group (see e.g., Scheme 3).

As used herein the term “GNeosome”, used alone or in combination with other terms, refers to a conjugate comprising a lipsosome and a guanidinylated neomycin group, or a derivative thereof. In some embodiments, the GNeosome is a conjugate consisting of a liposome, a guanidinylated neomycin group, or a derivative thereof. In some embodiments, the GNeosome is a conjugate comprising a liposome, a guanidinylated neomycin group, and a therapeutic agent. In some embodiments, the GNeosome is a conjugate consisting of a liposome, a guanidinylated neomycin group, and a therapeutic agent. In some embodiments, the guanidinylated neomycin group is coupled to an exterior surface of the liposome. In some embodiments, the therapeutic agent is enclosed within the liposome. In some embodiment, the therapeutic agent is enclosed within the liposome and the guanidinylated neomycin group is coupled to an exterior surface of the liposome.

As used herein the term “plain liposome” refers to a liposome that is not coupled to a guanidinylated neomycin group, or a derivative thereof.

### Compounds

The present application provides, inter alia, a compound of Formula I:

A-B-C I

or a pharmaceutically acceptable salt thereof, wherein:

A is a guanidinylated neomycin derivative;

B is a linker group; and

C is a phospholipid, a fatty acid or a fatty acid group.

In some embodiments, A is a guanidinylated neomycin derivative of the following formula:

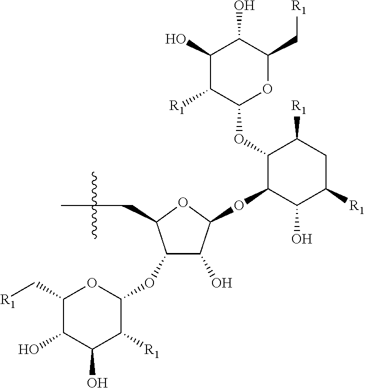

wherein:

R₁ is a guanidine or guanidinium group; and

[symbol] indicates the bond between A and B of Formula I.

In some embodiments, the guanidine group is an N-protected guanidine. In some embodiments, the N-protected guanidine group is of the following formula:

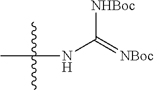

wherein Boc is tert-butoxycarbonyl.

In some embodiments, the guanidinium group is of the following formula:

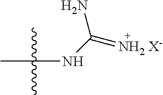

wherein X⁻ is an anion.

In some embodiments, the guanidinium group is of the following formula:

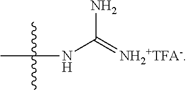

In some embodiments, B is a linker group comprising one or more alkylene groups. In some embodiments, each of the one or more alkylene groups is an independently selected C₁₋₃₀ alkylene group. In some embodiments, each of the one or more alkylene groups is an independently selected C₁₋₁₀ alkylene group. In some embodiments, each of the one or more alkylene groups is an independently selected C₁₋₆ alkylene group.

In some embodiments, B is a linker group comprising one or more amide groups. In some embodiments, each of the one or more amide groups is a group having the formula —(NHCO)—.

In some embodiments, B is a linker group comprising one or more alkyleneoxy groups. In some embodiments, each of the one or more alkyleneoxy groups is independently a group having the formula —(O-alkylene)-. In some embodiments, each of the one or more alkyleneoxy groups is independently a group having the formula —(O—C₁₋₆ alkylene)-. In some embodiments, each of the one or more alkyleneoxy groups is a group having the formula —(OCH₂CH₂)— or —(OCH₂)—.

In some embodiments, B is a linker group comprising one or more heteroarylene groups. As used herein, “heteroarylene” refers to a divalent, monocyclic or polycyclic aromatic heterocycle having at least one heteroatom ring member selected from sulfur, oxygen, and nitrogen. In some embodiments, the heteroarylene ring has 1, 2, 3, or 4 heteroatom ring members independently selected from nitrogen, sulfur and oxygen. In some embodiments, any ring-forming N in a heteroarylene moiety can be an N-oxide. In some embodiments, the heteroarylene has 5-10 ring atoms and 1, 2, 3 or 4 heteroatom ring members independently selected from nitrogen, sulfur and oxygen. In some embodiments, the heteroarylene has 5-6 ring atoms and 1 or 2 heteroatom ring members independently selected from nitrogen, sulfur and oxygen. In some embodiments, each of the one or more heteroarylene groups is an independently selected 5-6 membered heteroarylene group. In some embodiments, B is a linker group comprising one heteroarylene group. In some embodiments, B is a linker group comprising one 5-6 membered heteroarylene group. In some embodiments, B is a linker group comprising a heteroarylene group selected from the group consisting of:

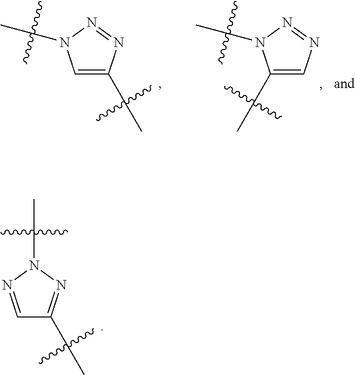 [SMILES: *c1cn(*)nn1 ; *c1cnnn1* ; *c1cnn(*)n1]

In some embodiments, B is a linker comprising a heteroarylene group which is:

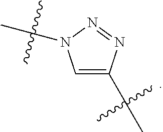 [SMILES: *c1cn(*)nn1]

In some embodiments, B is a linker group comprising one or more amine groups. In some embodiments, each of the one or more amine groups is independently a group having the formula —NR—, wherein R is hydrogen or a C₁₋₁₀ alkyl. In some embodiments, each of the one or more amine groups is independently a group having the formula —NH—. In some embodiments, B is a linker group comprising one amine group. In some embodiments, B is a linker group comprising one amine group having the formula —NH—.

In some embodiments, B is a linker group comprising a linker selected from the group consisting of one or more alkylene groups, one or more amide groups, one or more alkyleneoxy groups, one or more heteroaryl groups, one or more amine groups, or any combination thereof.

In some embodiments, B is a linker group comprising a linker selected from the group consisting of one or more alkylene groups, one or more amide groups, one or more alkyleneoxy groups, one or more heteroaryl groups, one or more amine groups, or any combination thereof.

In some embodiments, B is a linker group comprising one or more C₁₋₁₀ alkylene groups, one or more —(OCH₂CH₂)— or —(OCH₂)— groups, one amide group, one —NH— group, and one 5-6 membered heteroaryl group selected from the group consisting of:

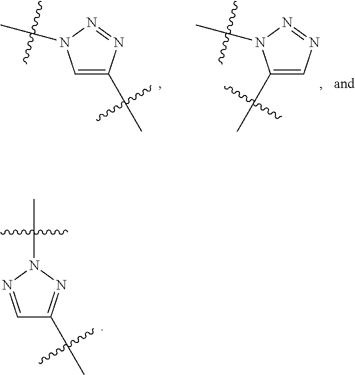 [SMILES: *c1cn(*)nn1 ; *c1cnnn1* ; *c1cnn(*)n1]

In some embodiments, B is a linker group comprising one or more C₁₋₁₀ alkylene groups, one or more —(OCH₂CH₂)— or —(OCH₂)— groups, one amide group, one —NH— group, and one 5-6 membered heteroaryl group which is:

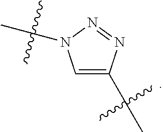 [SMILES: *c1cn(*)nn1]

In some embodiments, B is a linker group selected from the group of the following formulae:

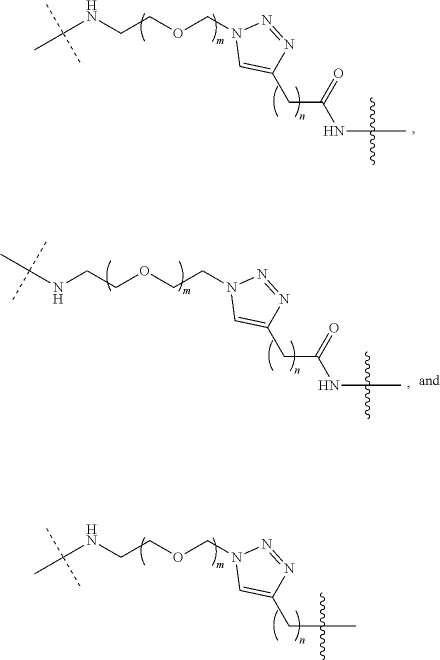

wherein:

m is an integer from 1 to 10;

n is an integer from 0 to 10;

[symbol] indicates the bond between A and B of Formula I; and

[symbol] indicates the bond between B and C of Formula I.

In some embodiments, m is an integer from 1 to 5. In some embodiments, m is an integer from 2 to 5. In some embodiments, m is an integer from 5 to 10. In some embodiments, m is 3.

In some embodiments, n is an integer from 0 to 5. In some embodiments, n is an integer from 5 to 10. In some embodiments, n is an integer from 0 to 3. In some embodiments, n is 0 or 3.

In some embodiments, C is a fatty acid or fatty acid group comprising one or more aliphatic groups comprising 4 to 50 carbon atoms. As used herein, the term “aliphatic group” refers to a straight or branched carbon chain (i.e., a non-cyclic carbon chain), which may be saturated or unsaturated (e.g., polyunsaturated). In some embodiments, the aliphatic group comprises 4 to 50 carbon atoms, for example, 4 to 50, 4 to 40, 4 to 30, 4 to 20, 4 to 15, 4 to 10, 10 to 50, 10 to 40, 10 to 30, 10 to 20, 10 to 15, 15 to 50, 15 to 40, 15 to 30, 15 to 20, 20 to 50, 20 to 40, 20 to 30, 30 to 50, 30 to 40, or 40 to 50 carbon atoms. In some embodiments, the aliphatic group is a saturated aliphatic group. In some embodiments, the aliphatic group is an unsaturated aliphatic group. In some embodiments, the aliphatic group is a straight chain aliphatic group. In some embodiments, the aliphatic group is a branched aliphatic group. Example fatty acids include, but are not limited to, butyric acid, stearoic acid, myristic acid, decanic acid, erucic acid, linoleic acid, lauric acid, oleic acid, palmitic acid, eicosapentaenic acid, docosahexaenoic acid, arachidonoic acid, docosatetraenoic acid, vaccenoic acid, and elaidoic acid. Example fatty acid groups include, but are not limited to butyroyl, stearoyl, myristoyl, decanoyl, erucoyl, linoleoyl, lauroyl, oleoyl, palmitoyl, eicosapentaenyl, docosahexaenoyl arachidonoyl, docosatetraenoyl, vaccenoyl, and elaidoyl.

In some embodiments, C is a phospholipid. As used herein, the term “phospholipid” refers to a group comprising one or more fatty acid groups comprising 4 to 50 carbon atoms and one or more phosphate groups. Example phospholipids include, but are not limited to, phosphatidylcholines (e.g., DDPC (1,2-didecanoyl-sn-glycero-3-phosphocholine), DEPC (1,2-dierucoyl-sn-glycero-3-phosphocholine), DLOPC (1,2-dilinoleoyl-sn-glycero-3-phosphocholine), DLPC (1,2-Dilauroyl-sn-glycero-3-phosphocholine), and POPC (1-palmitoyl-2-oleoyl-sn-glycero-3-phosphocholine), and the like), phosphatidylethanolamines (e.g., 1,2-dimyristoyl-sn-glycero-3-phosphoethanolamine (DMPE), 1,2-Dipalmitoyl-sn-glycero-3-phosphoethanolamine (DPPE), and the like). Additional examples of phospholipids may be found, for example, in Li et al, Asian Journal of Pharmaceutical Sciences, 2015, 10:81-98, the disclosure of which is incorporated by reference herein in its entirety.

In some embodiments, C is a phospholipid comprising one or more choline groups, one or more glycerophosphoric acid groups, and one or more fatty acid groups. In some embodiments, C comprises one fatty acid group. In some embodiments, C comprises two fatty acid groups. In some embodiments, each fatty acid group independently comprises from 4 to 50 carbon atoms, for example, 4 to 50, 4 to 40, 4 to 30, 4 to 20, 4 to 15, 4 to 10, 10 to 50, 10 to 40, 10 to 30, 10 to 20, 10 to 15, 15 to 50, 15 to 40, 15 to 30, 15 to 20, 20 to 50, 20 to 40, 20 to 30, 30 to 50, 30 to 40, or 40 to 50 carbon atoms. In some embodiments, at least one fatty acid group is a saturated fatty acid group. In some embodiments, at least one fatty acid group is an unsaturated fatty acid group. In some embodiments, at least one fatty acid group is a straight chain fatty acid group. In some embodiments, at least one fatty acid group is a branched fatty acid group. In some embodiments, the fatty acid group is selected from the group consisting of butyroyl, stearoyl, myristoyl, decanoyl, erucoyl, linoleoyl, lauroyl, oleoyl, and palmitoyl.

As used herein, the term “choline” or “choline group” refers to a group having the formula:

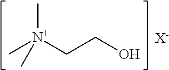

wherein X⁻ is an anion.

As used herein, the term “glycerophosphoric acid” refers to a group having the formula selected from the group consisting of:

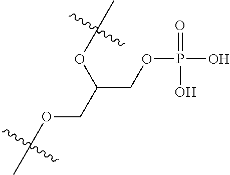 [SMILES: *OCC(COP(=O)(O)O)O*]

or a salt thereof.

In some embodiments, the glycerophosphoric acid group is coupled (e.g., covalently bonded) to the choline group. In some embodiments, the glycerophosphoric acid-choline moiety is selected from the group consisting of:

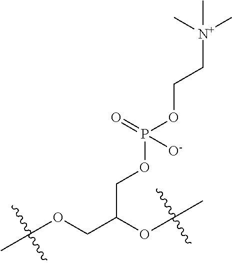 [SMILES: *OCC(COP(=O)([O-])OCC[N+](C)(C)C)O*]

wherein [symbol] indicates a bond to a fatty acid group.

In some embodiments, C is a phospholipid comprising one or more ethanolamine groups, one or more glycerophosphoric acid groups, and one or more fatty acid groups. In some embodiments, the glycerophosphoric acid is coupled (e.g., covalently bonded) to the ethanolamine group. In some embodiments, the glycerophosphoric acid-ethanolamine moiety is selected from the group consisting of:

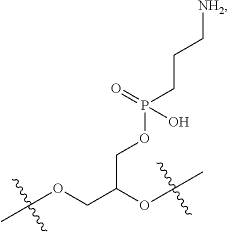 [SMILES: *OCC(COP(=O)(O)CCCN)O*]

wherein [symbol] indicates a bond to a fatty acid group.

In some embodiments C is a phosphatidylcholine. In some embodiments, C is a phosphatidylcholine selected from the group consisting of DDPC (1,2-didecanoyl-sn-glycero-3-phosphocholine), DEPC (1,2-dierucoyl-sn-glycero-3-phosphocholine), DLOPC (1,2-dilinoleoyl-sn-glycero-3-phosphocholine), DLPC (1,2-Dilauroyl-sn-glycero-3-phosphocholine), DMPC (1,2-dimyristoyl-sn-glycero-3-phosphocholine), DOPC (1,2-dioleoyl-sn-glycero-3-phosphocholine), DPPC (1,2-dipalmitoyl-sn-glycero-3-phosphocholine), DSPC (1,2-distearoyl-sn-glycero-3-phosphocholine), Milk Sphingomyelin MPPC (1-myristoyl-2-palmitoyl-sn-glycero 3-phosphocholine), MSPC (1-myristoyl-2-stearoyl-sn-glycero-3-phosphocholine), PMPC (1-palmitoyl-2-myristoyl-sn-glycero-3-phosphocholine, POPC (1-palmitoyl-2-oleoyl-sn-glycero-3-phosphocholine), PSPC (1-palmitoyl-2-stearoyl-sn-glycero-3-phosphocholine), SMPC (1-stearoyl-2-myristoyl-sn-glycero-3-phosphocholine), SOPC (1-stearoyl-2-oleoyl-sn-glycero-3-phosphocholine), and SPPC (1-stearoyl-2-palmitoyl-sn-glycero-3-phosphocholine). In some embodiments, C is a phosphatidylcholine selected from the group consisting of POPC (1-palmitoyl-2-oleoyl-sn-glycero-3-phosphocholine), DOPC (1,2-dioleoyl-sn-glycero-3-phosphocholine), and DOPE (1,2-dioleoyl-sn-glycero-3-phosphoethanolamine), or any combination thereof. In some embodiments, C is a phosphatidylcholine selected from the group consisting of POPC (1-palmitoyl-2-oleoyl-sn-glycero-3-phosphocholine), DOPC (1,2-dioleoyl-sn-glycero-3-phosphocholine), and DOPE (1,2-dioleoyl-sn-glycero-3-phosphoethanolamine).

In some embodiments, C is a phospholipid, a fatty acid or a fatty acid group selected from the following formulae:

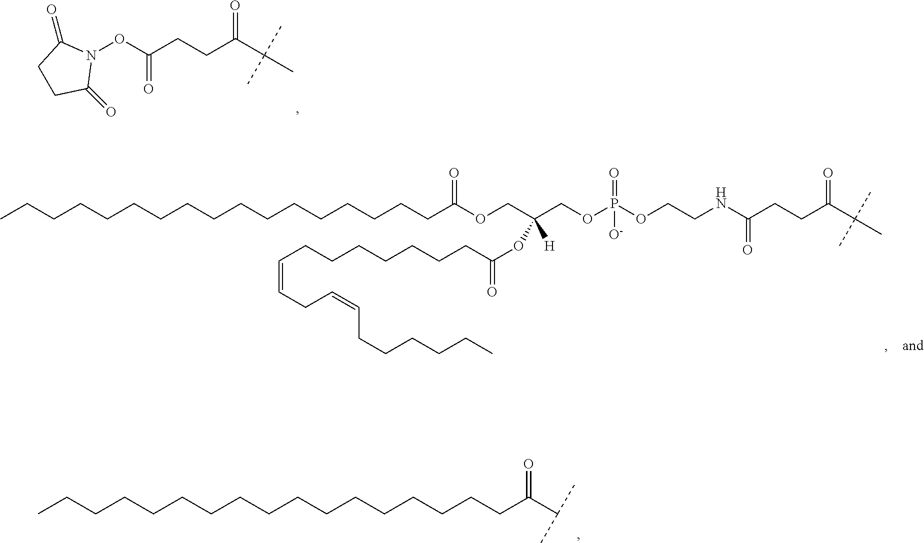

wherein [symbol] indicates the bond between B and C of Formula I.

In some embodiments, the compound of Formula I is selected from the group consisting of:

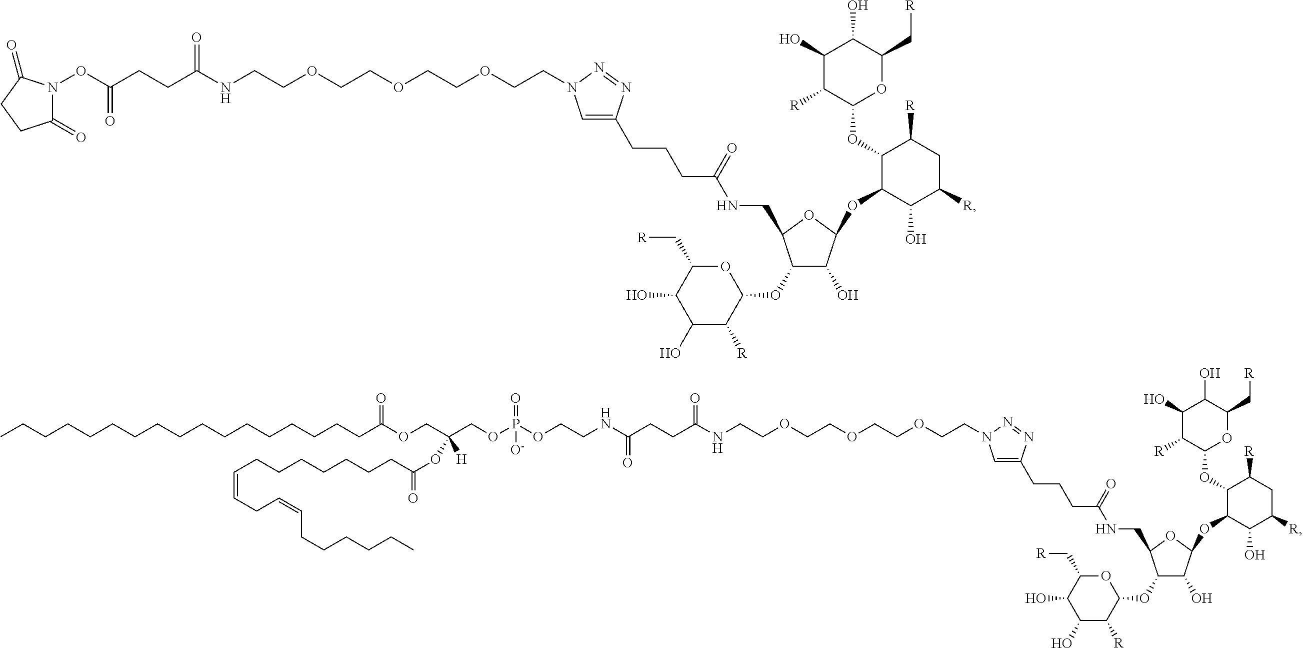

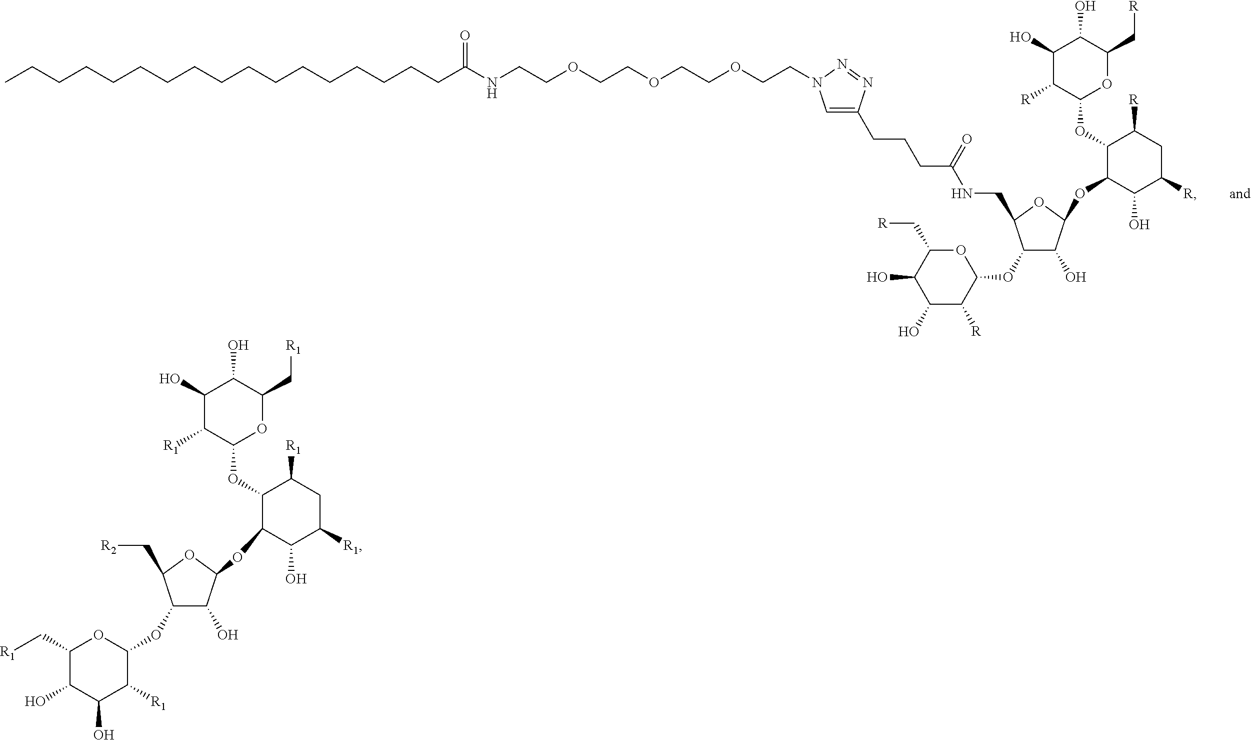

wherein:

each R is

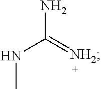

each R₁ is

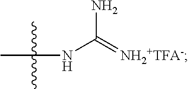

and

each R₂ is

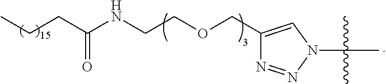

In some embodiments, the guanidinylated neomycin derivative is selected from the group consisting of a stearyl-GNeo, di-oleyl-GNeo, an NETS-protected (i.e., N-hydroxysuccinimide) GNeo group, and a phosphatidylcholine-GNeo, or a pharmaceutically acceptable salt thereof.

The term “guanidinylated neomycin derivative” as used herein refers to derivatives of neomycin in which one or more of the ammonium groups have been converted into guanidinium groups. In some cases, all of the ammonium groups can be converted into guanidinium groups. For example, guanidinylated neomycin (GNeo) may contain six positively charged guanidinium groups in place of the naturally occurring amino groups on the three monosaccharide units and the one cyclitol that make up the antibiotic. Additional glycosides may be substituted for the neomycin group (i.e. a guanidinylated aminoglycoside). Example guanidinylated aminoglycosides include, but are not limited to guanidino-amikacin, guanidino-gentamicin, guanidino-kanamycin, guanidino-neomycin, guanidino-netilmicin, guanidino-O-2,6-diamino-2,6-dideoxy-beta-L-idopyranosyl-(1 to 3)-O-beta-D-ribofuranosyl-(1 to 5)-O-[2-amino-2-deoxy-alpha-D-glucopyranosyl-(1 to 4)]-2-deoxystreptamine, guanidino-paramycin, guanidino-streptomycin, and guanidino-tobramycin.

The term “coupled” as used herein includes both covalent and noncovalent bonding of two or more moieties. In some cases, the term coupled can include covalent or noncovalent bonding which occurs directly between the moieties or optionally via one or more linkers. In some embodiments, the lipsome is directed bonded to the guanidinylated neomycin group. In some embodiments, the guanidinylated neomycin group is directly coupled to an exterior surface of the liposome (i.e., directed bonded to an exterior surface of the liposome).

### Conjugates and Methods of Treatment

The present application further provides a conjugate, comprising a compound provided herein and a liposome. In some embodiments, the liposome is a phospholipid liposome. In some embodiments, the liposome is a phosphatidylcholine liposome. In some embodiments, phosphatidylcholine liposome is prepared from a phosphatidylcholine selected from the group consisting of DDPC (1,2-didecanoyl-sn-glycero-3-phosphocholine), DEPC (1,2-dierucoyl-sn-glycero-3-phosphocholine), DLOPC (1,2-dilinoleoyl-sn-glycero-3-phosphocholine), DLPC (1,2-Dilauroyl-sn-glycero-3-phosphocholine), DMPC (1,2-dimyristoyl-sn-glycero-3-phosphocholine), DOPC (1,2-dioleoyl-sn-glycero-3-phosphocholine), DPPC (1,2-dipalmitoyl-sn-glycero-3-phosphocholine), DSPC (1,2-distearoyl-sn-glycero-3-phosphocholine), Milk Sphingomyelin MPPC (1-myristoyl-2-palmitoyl-sn-glycero 3-phosphocholine), MSPC (1-myristoyl-2-stearoyl-sn-glycero-3-phosphocholine), PMPC (1-palmitoyl-2-myristoyl-sn-glycero-3-phosphocholine, POPC (1-palmitoyl-2-oleoyl-sn-glycero-3-phosphocholine), PSPC (1-palmitoyl-2-stearoyl-sn-glycero-3-phosphocholine), SMPC (1-stearoyl-2-myristoyl-sn-glycero-3-phosphocholine), SOPC (1-stearoyl-2-oleoyl-sn-glycero-3-phosphocholine), and SPPC (1-stearoyl-2-palmitoyl-sn-glycero-3-phosphocholine). In some embodiments, phosphatidylcholine liposome is prepared from a phosphatidylcholine selected from the group consisting of POPC (1-palmitoyl-2-oleoyl-sn-glycero-3-phosphocholine), DOPC (1,2-dioleoyl-sn-glycero-3-phosphocholine), and DOPE (1,2-dioleoyl-sn-glycero-3-phosphoethanolamine), or any combination thereof. In some embodiments, phosphatidylcholine liposome is prepared from a phosphatidylcholine selected from the group consisting of POPC (1-palmitoyl-2-oleoyl-sn-glycero-3-phosphocholine), DOPC (1,2-dioleoyl-sn-glycero-3-phosphocholine), and DOPE (1,2-dioleoyl-sn-glycero-3-phosphoethanolamine).

In some embodiments, the liposome comprises a cholesterol. In some embodiments, the cholesterol is derived from a natural source, for example, an animal source (e.g., beef, pork, poultry, fish, shellfish), animal fat, dairy products (e.g., milk, cheese, and the like), and eggs.

In some embodiments, the liposome comprises a group selected from the group consisting of a POPC group (1-palmitoyl-2-oleoyl-sn-glycero-3-phosphocholine) derivative, a DOPC group (1,2-dioleoyl-sn-glycero-3-phosphocholine), and a cholesterol group, or any combination thereof.

In some embodiments, the conjugate is prepared by reacting a compound provided herein (e.g., a compound of Formula I) with a phospholipid to form the conjugate (i.e., the liposome). In some embodiments, the conjugate is prepared by reacting a guanidinylatedglycoside provided herein (e.g., guanidinylated neomycin) with a phospholipid provided herein to form the conjugate (i.e., the liposome). In some embodiments, the phospholipid is selected from the group consisting of POPC (1-palmitoyl-2-oleoyl-sn-glycero-3-phosphocholine), DOPC (1,2-dioleoyl-sn-glycero-3-phosphocholine), cholesterol, DOPE (1,2-dioleoyl-sn-glycero-3-phosphoethanolamine), or any combination thereof.

In some embodiments, the conjugate comprises a compound provided herein (e.g., a compound of Formula I, or a pharmaceutically acceptable salt thereof) which is coupled e.g., covalently bonded) to a surface of the liposome. In some embodiments, the conjugate comprises a compound provided herein which is coupled to the exterior surface of the liposome. In some embodiments, the conjugate comprises a compound provided herein which is covalently bonded to the exterior surface of the liposome. In some embodiments, the phospholipid is a phosphatidylcholine provided herein.

In some embodiments, the conjugate comprises a guanidinylated neomycin derivative selected from the group consisting of stearyl-GNeo, di-oleyl-GNeo, an NHS-protected (i.e., N-hydroxysuccinimide) GNeo group, and a phosphatidylcholine-GNeo, or a pharmaceutically acceptable salt thereof.

In some embodiments, the conjugate is a liposomal-GNeo conjugate (i.e., a GNeosome). In some embodiments, the conjugate provided herein comprises from about 0.5 to about 5% guanidinylated neomycin, for example, about 0.5 to about 5%, about 0.5 to about 4%, about 0.5 to about 3%, about 0.5 to about 2%, about 0.5 to about 1%, about 1 to about 5%, about 1 to about 4%, about 1 to about 3%, about 1 to about 2%, about 2 to about 5%, about 2 to about 4%, about 2 to about 3%, about 3 to about 5%, about 3 to about 4%, or about 4 to about 5%. In some embodiments, the conjugate provided herein comprises from about 0.5 to about 2% guanidinylated neomycin. In some embodiments, the conjugate provided herein comprises from about 0.5 to about 1% guanidinylated neomycin. In some embodiments, the conjugate provided herein comprises from about 1 to about 2% guanidinylated neomycin.

In some embodiments, the conjugate comprises one or more of POPC, DOPC, DOPE, cholesterol, and a guanidinylated aminoglycoside (e.g., guanidinylated neomycin). For example, a conjugate can include POPC and a guanidinylated aminoglycoside; DOPE and a guanidinylated aminoglycoside; a combination of DOPE and DOPC and a guanidinylated aminoglycoside; or a combination of DOPE, DOPC, and cholesterol, and a guanidinylated aminoglycoside.

In some embodiments, the conjugate is selected from the group consisting of POPC:Stearyl-GNeo, DOPC:Stearyl-GNeo, DOPC:DOPE:Stearyl-GNeo, DOPC:DOPE:Cholesterol:Stearyl-GNeo.

In some embodiments, the POPC:Stearyl-GNeo conjugate comprises a ratio of POPC:Stearyl-GNeo of from about 50:1 to about 150:1, for example, about 50:1, about 75:1, about 100:1, about 125:1, or about 150:1. In some embodiments, the ratio of POPC:Stearyl-GNeo is about 100:1.

In some embodiments, the ratio of DOPC:Stearyl-GNeo conjugate is from about 50:1 to about 150:1, for example, about 50:1, about 75:1, about 100:1, about 125:1, or about 150:1. In some embodiments, the ratio of POPC:Stearyl-GNeo is about 100:0.9.

In some embodiments, the DOPC:DOPE:Stearyl-GNeo conjugate comprises a ratio of DOPC:Stearyl-GNeo of from about 70:1 to about 90:1, for example, about 70:1, about 75:1, about 80:1, about 85:1, or about 90:1. In some embodiments, the DOPC:DOPE:Stearyl-GNeo conjugate comprises a ratio of DOPE:Stearyl-GNeo of from about 10:1 to about 20:1, for example, about 10:1, about 12:1, about 15:1, about 18:1, or about 20:1. In some embodiments, the ratio of DOPC:DOPE:Stearyl-GNeo is about 85:15:0.9.

In some embodiments, the DOPC:DOPE:Cholesterol:Stearyl-GNeo conjugate comprises a ratio of DOPC:Stearyl-GNeo of from about 60:1 to about 80:1, for example about 60:1, about 65:1, about 70:1, about 75:1, or about 80:1. In some embodiments, the DOPC:DOPE:Cholesterol:Stearyl-GNeo conjugate comprises a ratio of DOPE:Stearyl-GNeo of from about 5:1 to about 15:1, for example, about 5:1, about 10:1, or about 15:1. In some embodiments, the DOPC:DOPE:Cholesterol:Stearyl-GNeo conjugate comprises a ratio of Cholesterol:Stearyl-GNeo of from about 10:1 to about 20:1, for example, about 10:1, about 15:1, or about 20:1. In some embodiments, the ratio of is about 73:11:16:0.9.

In some embodiments, the conjugate further comprises one or more therapeutic agents. In some embodiments, the therapeutic agent is selected from the group consisting of an anti-inflammatory agent, a peptide, a protein, an enzyme, and a nucleic acid.

Additional conjugates that may be useful in the methods provided herein may be found, e.g., in U.S. Pat. No. 8,071,535, U.S. Patent Publication No. U.S. 2012/0189601, International Publication WO 2014/159878, and International Publication WO 2005/025513 all of which are incorporated by reference in their entirety.

The present application further provides guanidinylated neomycin conjugates (i.e., GNeosomes) for the treatment of lysosomal storage disorders, central nervous system (CNS) disorders, or neurological disorders. In some embodiments, the methods comprising treating a disease in a patient in need thereof, wherein the disease is selected from the group consisting of a lysosomal storage disorder, a central nervous system (CNS) disorder, or a neurological disorder. As used herein, the term “patient,” refers to any animal, including mammals. For example, mice, rats, other rodents, rabbits, dogs, cats, swine, cattle, sheep, horses, primates, and humans. In some embodiments, the patient is a human. In some embodiments, the method comprises administering to the patient a therapeutically effective amount of a compound or conjugate provided herein.

Clinical applications include enzyme replacement therapy for disorders in which a cell is missing an enzyme or polypeptide. Specific examples include lysosomal storage diseases, congenital disorders of glycosylation, and metabolic disorders characterized by missing or reduced enzyme activity in the cytoplasm. Non-limiting examples of lysosomal storage diseases include: Activator Deficiency; Alpha-mannosidosis; Aspartylglucosaminuria; Cholesteryl ester storage disease; Chronic Hexosaminidase A Deficiency; Cystinosis; Danon disease; Fabry disease; Farber disease; Fucosidosis; Galactosialidosis; Gaucher disease; GM1 gangliosidosis; I-Cell disease; Infantile Free Sialic Acid Storage Disease; Juvenile Hexosaminidase A deficiency; Krabbe disease; Metachromatic Leukodystrophy; Mucopolysaccharidoses disorders (e.g., Pseudo-Hurler polydystrophy; Hurler Syndrome; Scheie syndrome; Hurler-Scheie syndrome; Hunter syndrome; Sanfilippo syndrome type A; Sanfilippo syndrome type B; Sanfilippo syndrome type C; Sanfilippo syndrome type D; Morquio type A; Morquio type B; Maroteaux-Lamy; Sly syndrome; and Natowicz syndrome Hyaluronidase deficiency); Multiple sulfatase deficiency; Niemann-Pick disease; Neuronal Ceroid Lipofuscinoses (e.g, CLN6 disease; Batten-Spielmeyer-Vogt/Juvenile NCL/CLN3 disease; Finnish Variant/Late Infantile CLN5; Jansky-Bielschowsky disease/Late infantile CLN2/TPP1 Disease; Kufs/Adult-onset NCL/CLN4 disease; Northern Epilepsy/variant Late Infantile CLN8; Santavuori-Haltia/Infantile CLN1/PPT disease; and β-mannosidosis); Pompe disease; Pycnodysostosis; Sandhoff disease; Schindler disease; Salla disease; Tay-Sachs; and Wolman disease.

In addition, a compound or conjugate provided herein can be used to treat one or more CNS or neurological disorders such as Alzheimer's disease, Parkinson's disease, cerebrovascular disorders, frontotemporal dementia, personality disorders, cognition disorders, motor dysfunction, eating disorders, sleep disorders, affective disorders, anxiety disorders, schizophrenia, brain tumors, ataxia, bovine spongiform encephalopathy, West Nile virus encephalitis, Neuro-AIDS, brain injury, spinal cord injury, and multiple sclerosis.

### Synthesis

As will be appreciated, the compounds provided herein, including salts thereof, can be prepared using known organic synthesis techniques and can be synthesized according to any of numerous possible synthetic routes.

Guanidinylated-neomycin compounds provided herein, and pharmaceutically acceptable salts thereof can be prepared, for example, using methods analogous to those shown below in Scheme 1 for the preparation of Compound 4 (i.e., Stearyl-GNeo), by substituting the appropriate starting materials.

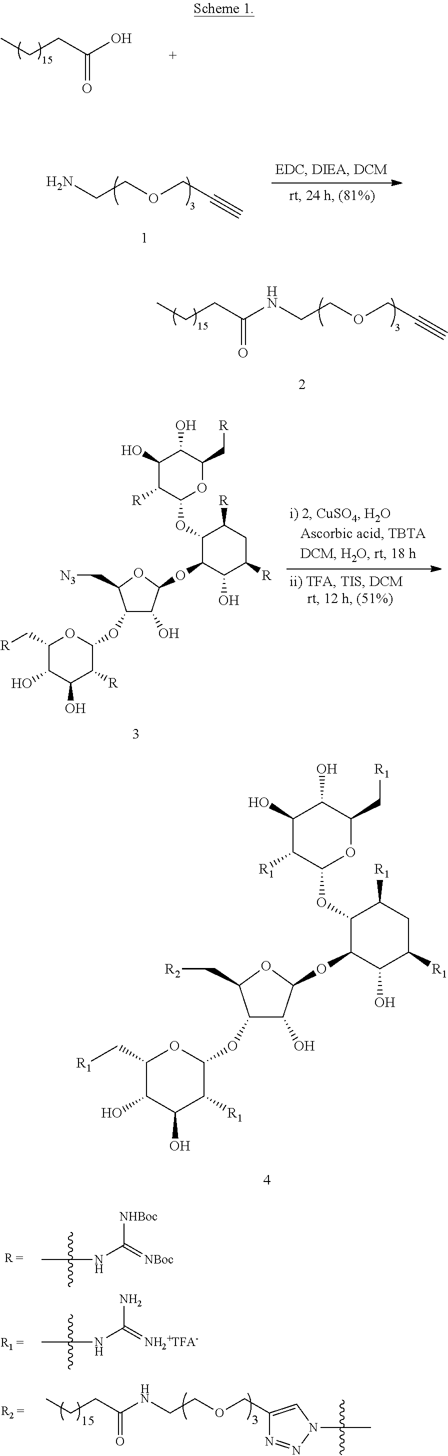

The guanidinylated neomycin liposome conjugates (i.e., GNeosomes) provided herein may be prepared, for example, using methods analogous to those shown below in Scheme 2. For example, fabrication of guanidinoglycoside-decorated liposomal assemblies can be accomplished in several ways, as shown in Scheme 2: a) Modification of surface amines on phosphatidylethanolamine (PE)-containing liposomes via NHSactivated-esters of guanidinoglycoside derivatives, b) Fabrication of mixed liposomes with phospholipids, which are conjugated to guanidinoglycosides, and c) Fabrication of mixed liposomes with amphiphilic guanidinoglycoside derivatives of fatty acids.

Prototypical (and non-limiting) structures of GNeo-phospholipid conjugates and their building blocks are shown below in Scheme 3. The synthesis of these compounds and related reagents follows procedures previously reported by Tor and coworkers and relies on a modular and orthogonal synthetic approach. The penultimate step is typically a mild Click reaction connects the azide-containing linkers and the alkyne-containing and fully protected guanidinoneomycin (e.g., GNeo-Alk with protected guanidine groups). The last step is a removal of the Boc protecting groups using acid (e.g. anhydrous trifluoroacetic acid). NHS activated esters have been shown to tolerate these conditions.

The linkers are prepared using standard procedures known in the art. Fully deprotected and NETS-activated derivatives (e.g., GNeo-NHS) have been used to successfully modify diverse proteins. GNeo-NHS can further be used for the preparation of GNeo-PE by a condensation reaction with PE or for modifying the outside surface of PE-containing liposomes, as shown above in Scheme 2, route a. Appropriate starting materials can be substituted for the preparation of GNeo-FA.

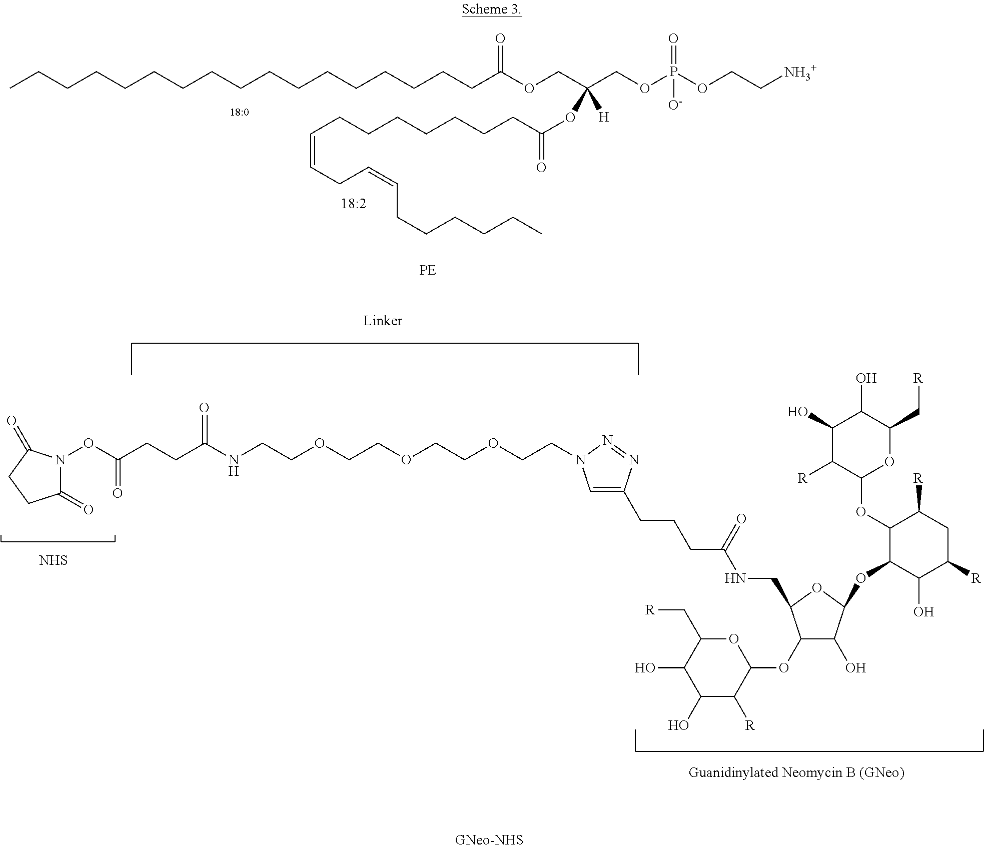

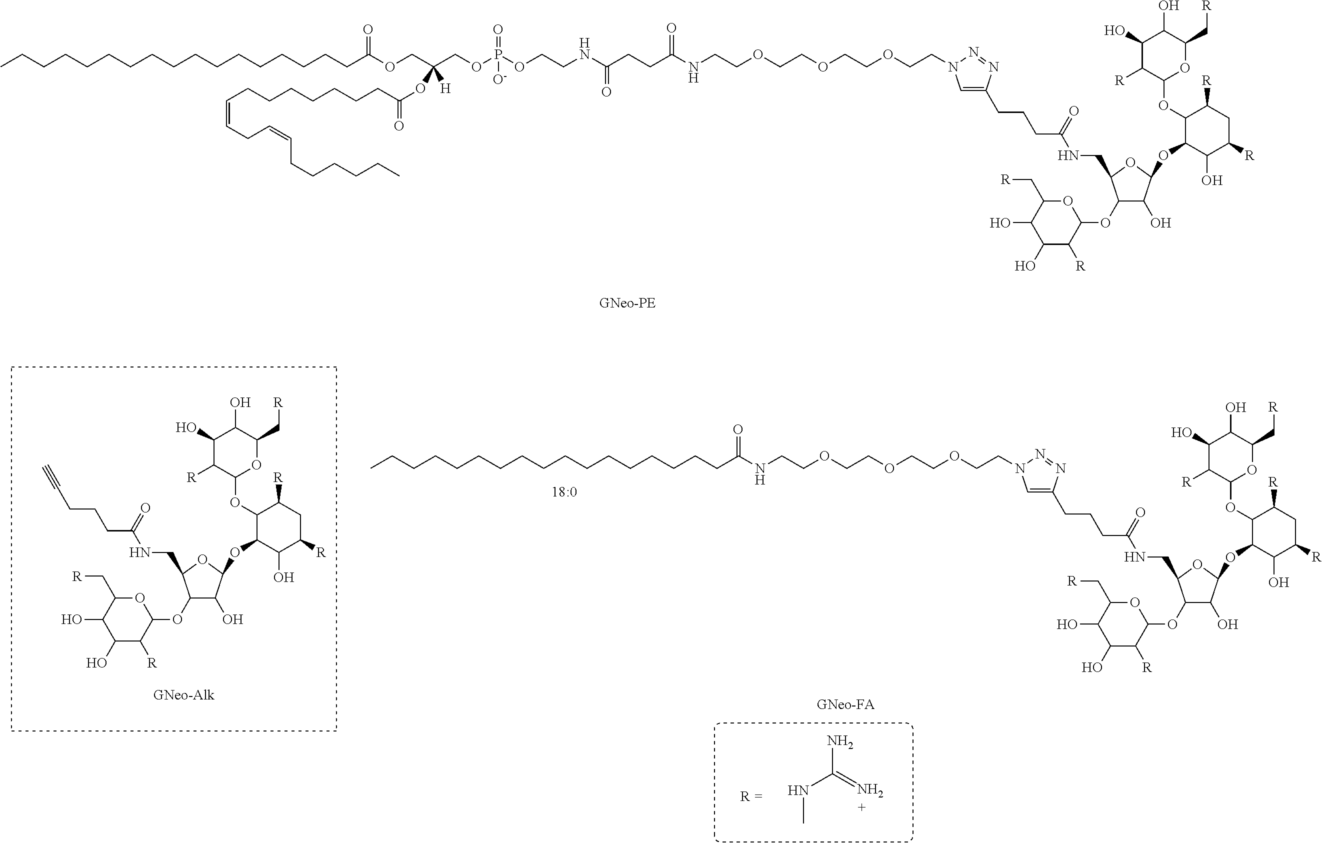

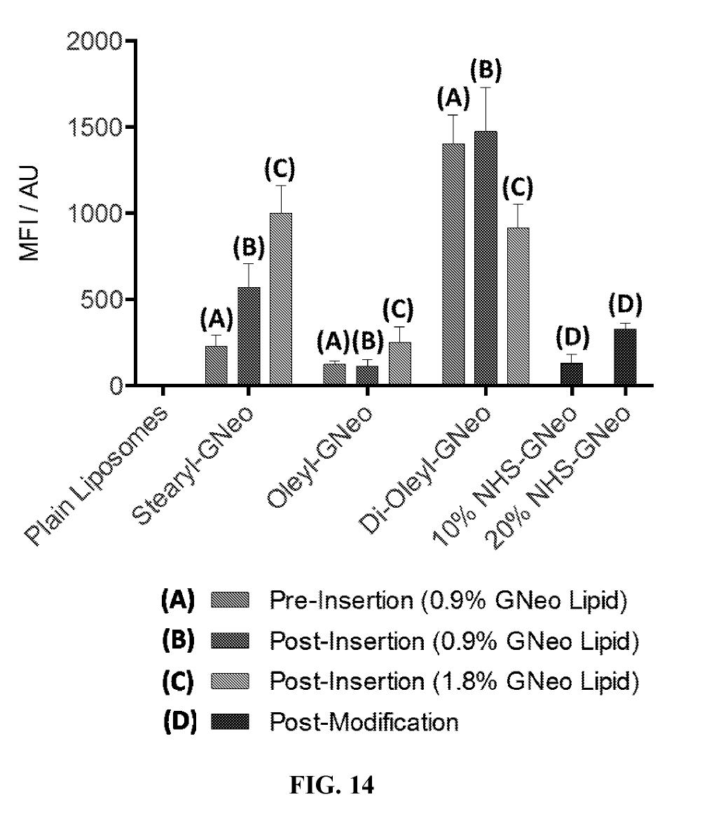

In some embodiments, the conjugate provided herein is prepared using a “pre-insertion” methodology as described herein (see e.g., Example 9 and FIG. 14). In some embodiments, the conjugate provided herein is prepared using a “post-insertion” methodology as described herein (see e.g., Example 9 and FIG. 14).

It will be appreciated by one skilled in the art that the processes described are not the exclusive means by which compounds provided herein may be synthesized and that a broad repertoire of synthetic organic reactions is available to be potentially employed in synthesizing compounds provided herein. The person skilled in the art knows how to select and implement appropriate synthetic routes. Suitable synthetic methods of starting materials, intermediates and products may be identified by reference to the literature, including reference sources such as: Advances in Heterocyclic Chemistry, Vols. 1-107 (Elsevier, 1963-2012); Journal of Heterocyclic Chemistry Vols. 1-49 (Journal of Heterocyclic Chemistry, 1964-2012); Carreira, et al. (Ed.) Science of Synthesis, Vols. 1-48 (2001-2010) and Knowledge Updates KU2010/1-4; 2011/1-4; 2012/1-2 (Thieme, 2001-2012); Katritzky, et al. (Ed.) Comprehensive Organic Functional Group Transformations, (Pergamon Press, 1996); Katritzky et al. (Ed.); Comprehensive Organic Functional Group Transformations II (Elsevier, 2^{nd} Edition, 2004); Katritzky et al. (Ed.), Comprehensive Heterocyclic Chemistry (Pergamon Press, 1984); Katritzky et al., Comprehensive Heterocyclic Chemistry II, (Pergamon Press, 1996); Smith et al., March's Advanced Organic Chemistry: Reactions, Mechanisms, and Structure, 6^{th} Ed. (Wiley, 2007); Trost et al. (Ed.), Comprehensive Organic Synthesis (Pergamon Press, 1991).

The reactions for preparing compounds described herein can be carried out in suitable solvents which can be readily selected by one of skill in the art of organic synthesis. Suitable solvents can be substantially non-reactive with the starting materials (reactants), the intermediates, or products at the temperatures at which the reactions are carried out, (e.g., temperatures which can range from the solvent's freezing temperature to the solvent's boiling temperature). A given reaction can be carried out in one solvent or a mixture of more than one solvent. Depending on the particular reaction step, suitable solvents for a particular reaction step can be selected by the skilled artisan.

Preparation of compounds described herein can involve the protection and deprotection of various chemical groups. The need for protection and deprotection, and the selection of appropriate protecting groups, can be readily determined by one skilled in the art. The chemistry of protecting groups can be found, for example, in T. W. Greene and P. G. M. Wuts, Protective Groups in Organic Synthesis, 3^{rd} Ed., Wiley & Sons, Inc., New York (1999).

Reactions can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), mass spectrometry, or by chromatographic methods such as high performance liquid chromatography (HPLC), liquid chromatography-mass spectroscopy (LCMS), or thin layer chromatography (TLC). Compounds can be purified by those skilled in the art by a variety of methods, including high performance liquid chromatography (HPLC) and normal phase silica chromatography.

The term “compound” as used herein is meant to include all stereoisomers, geometric isomers, tautomers, and isotopes of the structures depicted. Compounds herein identified by name or structure as one particular tautomeric form are intended to include other tautomeric forms unless otherwise specified.

Compounds provided herein also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone-enol pairs, amide-imidic acid pairs, lactam-lactim pairs, enamine-imine pairs, and annular forms where a proton can occupy two or more positions of a heterocyclic system, for example, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution.

All compounds, and pharmaceutically acceptable salts thereof, can be found together with other substances such as water and solvents (e.g. hydrates and solvates) or can be isolated.

In some embodiments, preparation of compounds can involve the addition of acids or bases to affect, for example, catalysis of a desired reaction or formation of salt forms such as acid addition salts.

Example acids can be inorganic or organic acids and include, but are not limited to, strong and weak acids. Some example acids include hydrochloric acid, hydrobromic acid, sulfuric acid, phosphoric acid, p-toluenesulfonic acid, 4-nitrobenzoic acid, methanesulfonic acid, benzenesulfonic acid, trifluoroacetic acid, and nitric acid. Some weak acids include, but are not limited to acetic acid, propionic acid, butanoic acid, benzoic acid, tartaric acid, pentanoic acid, hexanoic acid, heptanoic acid, octanoic acid, nonanoic acid, and decanoic acid.

Example bases include lithium hydroxide, sodium hydroxide, potassium hydroxide, lithium carbonate, sodium carbonate, potassium carbonate, and sodium bicarbonate. Some example strong bases include, but are not limited to, hydroxide, alkoxides, metal amides, metal hydrides, metal dialkylamides and arylamines, wherein; alkoxides include lithium, sodium and potassium salts of methyl, ethyl and t-butyl oxides; metal amides include sodium amide, potassium amide and lithium amide; metal hydrides include sodium hydride, potassium hydride and lithium hydride; and metal dialkylamides include lithium, sodium, and potassium salts of methyl, ethyl, n-propyl, iso-propyl, n-butyl, tert-butyl, trimethylsilyl and cyclohexyl substituted amides.

In some embodiments, the compounds provided herein, or salts thereof, are substantially isolated. By “substantially isolated” is meant that the compound is at least partially or substantially separated from the environment in which it was formed or detected. Partial separation can include, for example, a composition enriched in the compounds provided herein. Substantial separation can include compositions containing at least about 50%, at least about 60%, at least about 70%, at least about 80%, at least about 90%, at least about 95%, at least about 97%, or at least about 99% by weight of the compounds provided herein, or salt thereof. Methods for isolating compounds and their salts are routine in the art.

### Pharmaceutical Formulations

The present application further provides the manufacture and use of pharmaceutical compositions comprising a conjugate provided herein.

The phrase “pharmaceutically acceptable” is employed herein to refer to those ligands, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition, or vehicle, such as a liquid or solid filler, diluent, excipient, solvent or encapsulating material. As used herein the language “pharmaceutically acceptable carrier” includes buffer, sterile water for injection, solvents, dispersion media, coatings, antibacterial and antifungal agents, isotonic and absorption delaying agents, and the like, compatible with pharmaceutical administration. Each carrier must be “acceptable” in the sense of being compatible with the other ingredients of the formulation and not injurious to the patient. Some examples of materials which can serve as pharmaceutically acceptable carriers include: (1) sugars, such as lactose, glucose, and sucrose; (2) starches, such as corn starch, potato starch, and substituted or unsubstituted β-cyclodextrin; (3) cellulose, and its derivatives, such as sodium carboxymethyl cellulose, ethyl cellulose, and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil, and soybean oil; (10) glycols, such as propylene glycol; (11) polyols, such as glycerin, sorbitol, mannitol, and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations. In certain embodiments, pharmaceutical compositions provided herein are non-pyrogenic, i.e., do not induce significant temperature elevations when administered to a patient.

The term “pharmaceutically acceptable salt” refers to the relatively non-toxic, inorganic and organic acid addition salts of a conjugate provided herein. These salts can be prepared in situ during the final isolation and purification of a compound provided herein, or by separately reacting the conjugate in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, naphthylate, mesylate, glucoheptonate, lactobionate, laurylsulphonate salts, and amino acid salts, and the like. (See, for example, Berge et al. (1977) “Pharmaceutical Salts”, J. Pharm. Sci. 66: 1-19.)

In some embodiments, a conjugate provided herein may contain one or more acidic functional groups and, thus, is capable of forming pharmaceutically acceptable salts with pharmaceutically acceptable bases. The term “pharmaceutically acceptable salts” in these instances refers to the relatively non-toxic inorganic and organic base addition salts of a conjugate provided herein. These salts can likewise be prepared in situ during the final isolation and purification of the conjugate, or by separately reacting the purified conjugate in its free acid form with a suitable base, such as the hydroxide, carbonate, or bicarbonate of a pharmaceutically acceptable metal cation, with ammonia, or with a pharmaceutically acceptable organic primary, secondary, or tertiary amine. Representative alkali or alkaline earth salts include the lithium, sodium, potassium, calcium, magnesium, and aluminum salts, and the like. Representative organic amines useful for the formation of base addition salts include ethylamine, diethylamine, ethylenediamine, ethanolamine, diethanolamine, piperazine, and the like (see, for example, Berge et al., supra).

For administration by inhalation, the conjugates can be delivered in the form of an aerosol spray from a pressured container or dispenser that contains a suitable propellant, e.g., a gas such as carbon dioxide, or a nebulizer. Such methods include those described in U.S. Pat. No. 6,468,798. Additionally, intranasal delivery can be accomplished, as described in, inter alia, Hamajima et al., Clin. Immunol. Immunopathol., 88(2), 205-10 (1998) and U.S. Patent Publication Nos. 2008/0305077 and 2009/0047234, and International Patent Application No. WO 2008/049897. Liposomes (e.g., as described in U.S. Pat. No. 6,472,375), microencapsulation and nanoencapsulation can also be used. Biodegradable targetable microparticle delivery systems or biodegradable targetable nanoparticle delivery systems can also be used (e.g., as described in U.S. Pat. No. 6,471,996).

In some embodiments, the conjugates provided herein are formulated for intravenous administration. Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filter sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof.

The determination of a therapeutically effective dosage of a conjugate and/or therapeutic agent provided herein will be based on animal model studies, followed up by human clinical trials, and is guided by determining therapeutically effective dosages and nasal administration protocols that significantly reduce the occurrence or severity of the targeted disease symptoms or conditions in the patient. Suitable models in this regard include, for example, murine, rat, porcine, feline, non-human primate, and other accepted animal model subjects known in the art. Ultimately, the dosage of conjugate and/or therapeutic agent provided herein will be at the discretion of the attendant, physician or clinician. The dosage can also be adjusted by the individual physician in the event of any complication.

### Combination Therapies

One or more additional therapeutic agents such as, for example, anti-inflammatory agents, steroids, or immunosuppressants can be used in combination with the compounds of the present application for treatment of the diseases provided herein.

Example steroids include corticosteroids such as cortisone, dexamethasone, hydrocortisone, methylprednisolone, prednisolone, and prednisone.

Example anti-inflammatory compounds include aspirin, choline salicylates, celecoxib, diclofenac potassium, diclofenac sodium, diclofenac sodium with misoprostol, diflunisal, etodolac, fenoprofen, flurbiprofen, ibuprofen, ketoprofen, meclofenamate sodium, mefenamic acid, nabumetone, naproxen, naproxen sodium, oxaprozin, piroxican, rofecoxib, salsalate, sodium salicylate, sulindac, tolmetin sodium, and valdecoxib.

Example immunosuppressants include azathioprine, chlorambucil, cyclophosphamide, cyclosporine, daclizumab, infliximab, methotrexate, and tacrolimus.

Additional examples of therapeutic agents that can be used in combination with the conjugates provided herein for the treating of the diseases provided herein (e.g., a lysosomal storage disease) may be found, for example, in Kirkegaard, Expert Opinion on Orphan Drugs, 2013, 1(5):385-404, the disclosure of which is incorporated by reference herein in its entirety.

### EXAMPLES

The invention is further described in the following examples, which do not limit the scope of the invention described in the claims.

### General Methods

Materials obtained from commercial suppliers were used without further purification. Chemicals and reagents were obtained from Sigma Aldrich. DOPC (1,2-dioleoyl-sn-glycero-3-phosphocholine) and DOPE (1,2-dioleoyl-sn-glycero-3-phosphoethanolamine) were purchased from Avanti Polar Lipids. Deuterated NMR solvents were purchased from Cambridge Isotope Laboratories. PBS (Dulbecco's phosphate buffered saline), F-12 Nutrient Mixture (Ham), DMEM phenol red-free, Streptavidin-Cy3, Fluorescein di-β-D-galactopyranoside (FDG), LysoTracker® Green DND-26, LysoTracker® Deep Red, LysoSensor™ Dextran Blue/Yellow and nuclear stain Hoechst 33342 were purchased from Life Technologies (San Diego, Calif., USA). Trypsin/EDTA was purchased from VWR (Mediatech, Manassas, Va., USA). Costar 3524 (Corning) 24-well plates were used. 35 mm glass bottom culture dishes were purchased from MatTek (Ashland, Mass., USA).

NMR spectra were recorded on either a Varian Mercury 400 MHz or 500 MHz spectrometers. Mass spectra were recorded at the UCSD Chemistry and Biochemistry Mass Spectrometry Facility; low resolution mass spectrometry (LR-MS) analysis was performed on a Thermo LCQdeca mass spectrometer using electrospray ionization (ESI) as the ion source. An Agilent 6230 time of flight mass spectrometer (TOFMS) was employed for high resolution MS (HR-MS) analysis using ESI as the ion source.

Reversed phase HPLC purification (CLIPEUS, C₁₈, 5 μm, 10□250 mm, Higgins analytical) and analysis (Eclipse, XDB-C₁₈, 5 μm, 4.6□150 mm) were carried out on an Agilent 1200 series instrument. Fluorescence spectroscopy measurements have been performed on a Horiba fluorimeter. Flow-cytometry studies were performed on a BD FACSCalibur. Particle size (diameter, nm), polydispersity, and surface charge (zeta potential, mV) of the lipid vesicles were measured by dynamic light scattering (DLS) on a Zetasizer Nano ZS (model ZEN3600 from Malvern Instruments) and on a Wyatt Dynapro Nanostar (particle size). Confocal laser scanning microscopy was performed using a Nikon A1R inverted fluorescence microscope with z-stepping motor. Images were processed and analyzed using Nikon Imaging Software Elements and ImageJ (NIH).

Additional methodology, synthetic processes, and assays are described, e.g., in Appendices A, B, and C, which are attached hereto and are each incorporated by reference in their entirety.

### Stearyl-GNeo-Decorated Liposomes

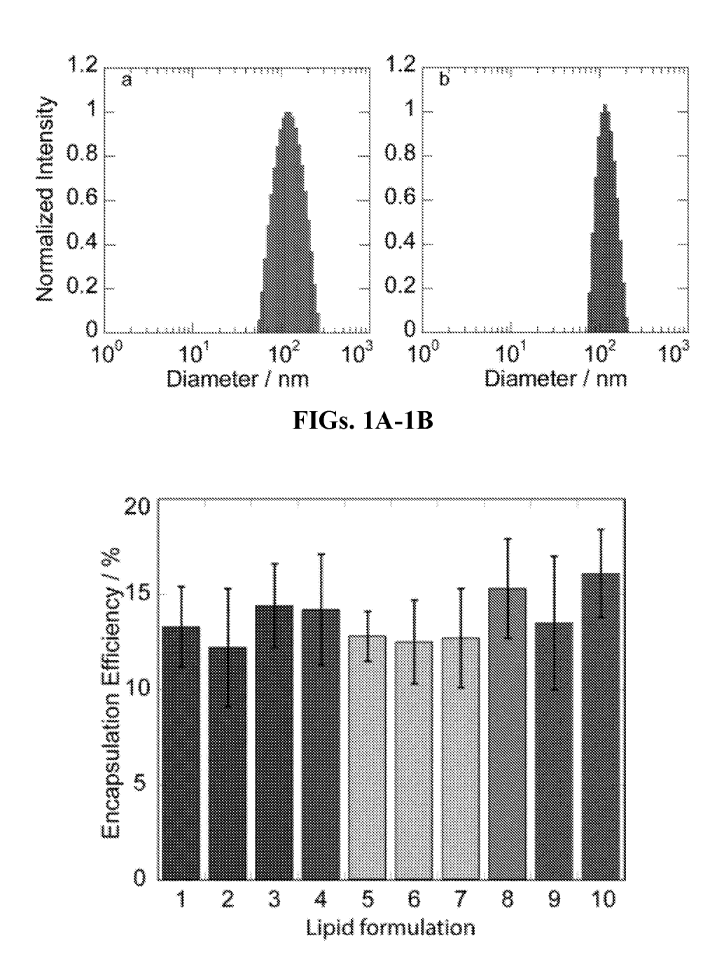

Stearyl-GNeo-decorated liposomes were prepared by rehydrating a lipid film [consisting of either POPC (1-palmitoyl-2-oleoyl-sn-glycero-3-phosphocholine), DOPC (1,2-dioleoyl-sn-glycero-3-phosphocholine), DOPC:DOPE (1,2-dioleoyl-sn-glycero-3-phosphoethanolamine) 85:15 or DOPC:DOPE:cholesterol, 73:11:16] with phosphate buffered saline (PBS, pH 7.4) and Stearyl-GNeo 4 (0.9 mol %), followed by sonication, freeze and thaw cycles and extrusion through 100 nm polycarbonate membranes. Plain liposomes were prepared in the same manner without 4. Dynamic light scattering analysis shows that the addition of Stearyl-GNeo results in a significant increase in zeta potential while the average size of the liposomes and their polydispersity remain comparable as shown below in Table 1 and in FIG. 1.²⁸

Lipid formulations were prepared as follows: Plain liposomes, DOPC:DOPE:Cholesterol 73:11:16; GNeosomes, DOPC:DOPE:Cholesterol:Stearyl-GNeo 73:11:16:0.9; DOTAP-M, DOPC:DOPE:Cholesterol:DOTAP 73:11:16:0.9; DOTAP-N, DOPC:DOPE:Cholesterol:DOTAP 73:11:16:5.4.

TABLE 1
Physiochemical Characterization of Lipid Vesicles.

| | Z-Average | | Z-potential |
|---|---|---|---|
| | (±SD)/nm | PDI (±SD) | (±SD)/mV |
| Plain liposomes | 136.0 (3.4) | 0.154 (0.030) | 0.44 (0.034) |
| GNeosomes | 125 (0.6) | 0.163 (0.023) | 27.6 (0.493) |
| DOTAP-M | 123.7 (1.8) | 0.153 (0.026) | 12.0 (0.321) |
| DOTAP-N | 115.3 (2.5) | 0.200 (0.017) | 33.2 (1.020) |

### Cell Culture

All cells were grown under an atmosphere of 5% CO₂ in air and 100% relative humidity. Wild-type Chinese hamster ovary cells (CHO-K1) were obtained from the American Type Culture Collection (CCL-61), and pgsA-745 cells were prepared as previously reported.^{31,51} CHO-K1 and pgsA-745 cells were grown in F-12 medium (Life Technologies) supplemented with fetal bovine serum (10% v/v), streptomycin sulfate (100 μg/mL), and penicillin G (100 Units/mL). Hep3B cell line was obtained from ATCC (HB-8064) and cultured in MEM (Invitrogen) supplemented with 10% fetal bovine serum (Gemini Bio-Products), nonessential amino acids, penicillin (100 Units/mL), and streptomycin (100 μg/mL). HEK293T cells were obtained from ATCC and maintained in Dulbecco's modified Eagle medium (DMEM) supplemented with 10% FBS, penicillin (100 Units/mL), and streptomycin (100 μg/mL).

### Encapsulation Efficiency

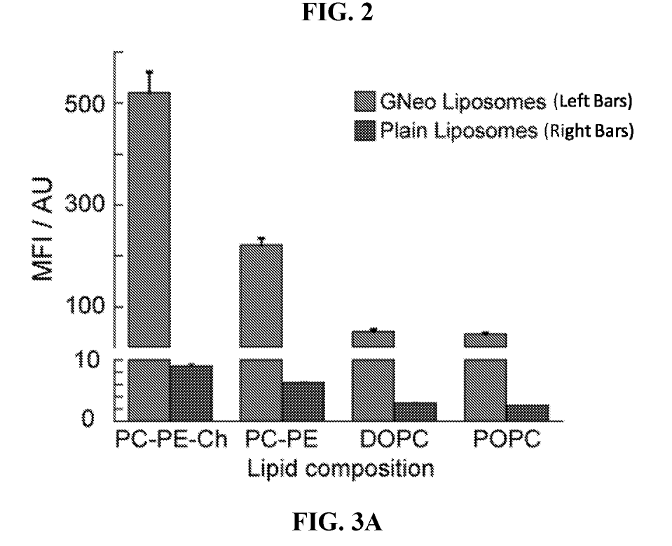

EE was calculated as the ratio between the fluorescence intensity (640/672) of 1 mL methanolic solution of lipid vesicles (0.1 mg/mL) before and after size exclusion purification. See FIG. 2.

### Cell Viability

CHO-K1 cells were seeded in a 96 wells plate at a density of 20000 cells per well. After growing overnight, the cells were treated with liposomes and GNeosomes at the indicated concentrations in serum free medium and incubated for 24 hours. Cells were washed and the growth medium was replaced. Cell Titer Blue (20 μL) was added to each well, and the cells were incubated for 4 hours at 37° C. Fluorescence was measured in a plate reader with excitation/emission wavelengths set at 530/580. Fluorescence intensity was normalized to that of untreated cells.

### Cellular Uptake

Wild-type CHO-K1, HEK293T, Hep3B and mutant pgsA cells were seeded onto 24-well tissue culture plates (100,000 cells/well, 0.5 mL; 250000 cells/well for Hep3B) and grown for 24 h to about 80% confluence. Cells were washed with PBS and incubated with 300 μL of the liposomal suspension diluted in growth medium containing 10% FBS to the desired concentration and incubated at 37° C. for one hour under an atmosphere of 5% CO₂. The cells were washed twice with 300 μL of PBS twice, detached with 100 μL of trypsin-EDTA (Life Technologies) at 37° C. for 5 min, diluted with PBS containing 1% BSA and analyzed by FACS.

### Evaluation of Clathrin-Dependent Endocytosis and Macropinocytosis of Liposomes

Cells were grown for 24 h as described above, washed with PBS and incubated with 400 mM sucrose or 5 mM amiloride for 1 hour or 10 minutes respectively. Cells were then washed with PBS and treated with 300 μL of the liposomal suspension diluted in growth medium to 300 μg/mL and incubated at 37° C. for one hour under an atmosphere of 5% CO₂. Cells were washed with PBS, detached with trypsin-EDTA and analyzed as described above.

### Evaluation of Uptake Dependency on Temperature

Cells were grown for 24 h as described above, washed with PBS and incubated for 30 minutes at 4° C. in F-12. Pre-cooled liposomes, diluted in F-12 to 300 μg/mL, were added to the cooled cells and incubated for 30 minutes at 4° C. Cells were washed, detached and analyzed as described above.

### Fluorescence Microscopy

CHO-K1 cells were grown for 24 h in 35 mm dishes equipped with a glass bottom coverslip coated with poly-D-lysine. Cells were washed with PBS and treated with 1.5 mL of liposomal suspension diluted in growth medium to 1 mg/mL and incubated at 37° C. for one hour under an atmosphere of 5% CO₂. Cells were then washed with PBS, stained with the appropriate dye (Hoechst, Lysotracker) and kept in DMEM phenol red-free medium for imaging.

### Ratiometric Analysis

Cells were grown in 35 mm dishes as above and incubated with 1.5 mL of LysoSensor™ Dextran Blue/Yellow or with GNeosomes (encapsulating LysoSensor™ Dextran Blue/Yellow) diluted in growth medium to 1 mg/mL at 37° C. for one hour under an atmosphere of 5% CO₂. Cells were then washed with PBS and kept in DMEM phenol red-free medium for imaging. Both “blue” and “green” exciting lasers were set at 405 nm. Images were processed and analyzed using Nikon Imaging Software Elements and ImageJ (NIH).

### Example 1. Compound 2

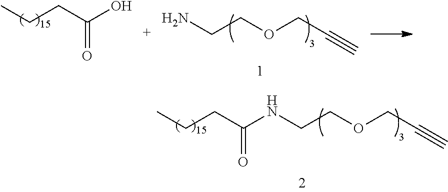

To a solution of stearic acid (250 mg, 0.87 mmol) in dichloromethane (DCM) (4 mL), was added EDC (222 mg, 1.2 mmol) and the solution was stirred at room temperature for 30 minutes. Compound 1 (109 mg, 0.58 mmol) and DIEA (103 μL, 0.58 mmol) were dissolved in DCM (2 mL) and added to the reaction. After stirring overnight at room temperature the reaction was diluted with DCM (30 mL) and washed with aqueous citric acid (5%, 30 mL) and brine. The organic phase was dried over sodium sulfate and evaporated. The residue was purified by flash chromatography (Hexane to 50% Hexane in Ethyl Acetate) to afford the desired compound as a white amorphous powder. Yield: 81%, 213 mg.

NMR (400 MHz, CDCl₃): δ 6.1 (s, 1H), 4.2 (d, J=2.4 Hz, 2H), 3.7 (m, 4H), 3.63 (m, 4H), 3.55 (t, J=4.9 Hz, 2H), 3.45 (m, 2H), 2.43 (t, J=2.4 Hz, 1H), 2.17 (t, J=7.4 Hz, 2H), 1.63 (m, 2H), 1.24-1.27 (s+m, 30H), 0.87 (t, J=6.7 Hz, 3H). ₁₃C NMR (126 MHz, CDCl₃): δ 173.31, 79.45, 74.69, 70.49, 70.33, 70.14, 69.96, 69.07, 58.41, 39.08, 36.75, 31.92, 29.70, 29.69, 29.67, 29.66, 29.64, 29.53, 29.40, 29.37, 29.35, 25.77, 22.70, 14.15. HRMS: Calculated [M+H]+ 454.3891, found: 454.3893.

### Example 2. Compound 4 (Stearyl-GNeo)

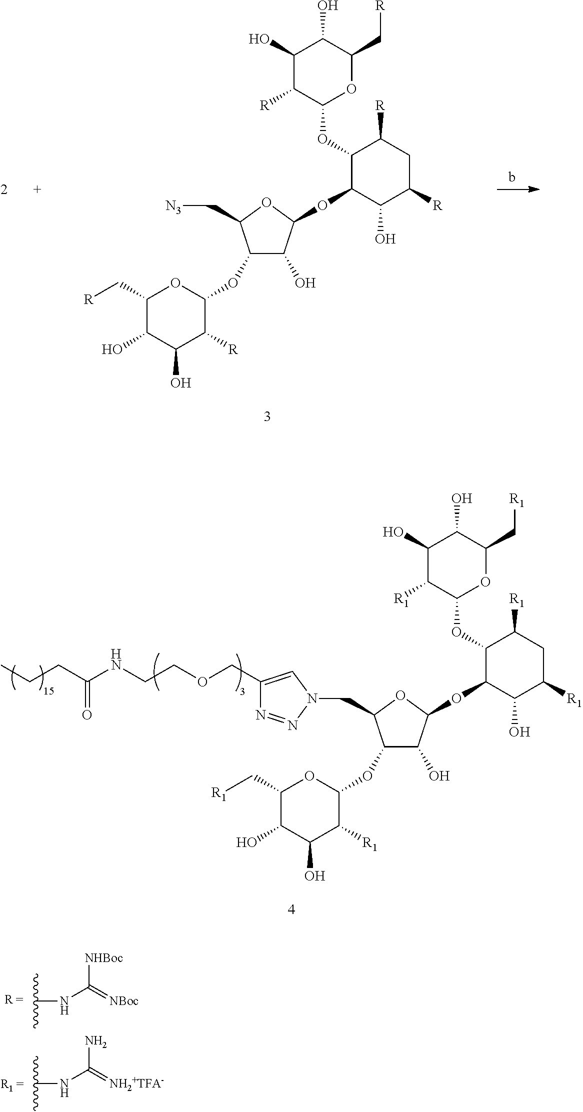

Compounds 2 (5.8 mg, 12.7 μmol) and 3 (30 mg, 14 μmol) were dissolved in DCM (200 μL) and TBTA (0.3 mg, 0.64 μmol) was added. CuSO₄.5H₂O (0.2 mg, 0.64 μmol) and sodium ascorbate (0.4 mg, 1.9 μmol) were dissolved in water (200 μL) and added to the organic solution. The mixture was vigorously stirred at room temperature for 18 hours and then diluted with DCM (5 mL) and water (5 mL). The organic phase was washed twice with aqueous EDTA (0.1 M, 5 mL), aqueous KCN (5%, 5 mL) and brine (5 mL). The organic phase was dried over sodium sulfate and evaporated. The residue was dissolved in DCM (0.5 mL) and triethylsilane (50 μL) and trifluoroacetic acid (0.5 mL) were added. The reaction was stirred 12 hours at room temperature, concentrated under vaccum and coevaporated three times with toluene. The residue was dissolved in 5% aqueous acetonitrile (600 μL) and purified on reversed phase HPLC to obtain the desired compound as an amorphous fluffy white powder. Yield: 51%, 13 mg (over two steps).

¹H NMR (400 MHz, D₂O): δ 7.95 (s, 1H), 5.69 (s, 1H), 5.02 (s, 1H), 4.99 (s, 1H), 4.69 (m, 4H), 4.41 (bs, 1H), 4.31 (bs, 1H), 4.24 (m, 1H), 4.08-4.06 (m, 2H), 3.75-3.29 (m, 30H), 2.14 (m, 3H), 1.64-1.48 (m, 4H), 1.18 (bs, 30H), 0.79 (t, J=6.2 Hz, 3H); ¹³C NMR (126 MHz, D₂O): δ 176.07, 163.02, 162.74, 162.46, 162.18, 157.50, 157.09, 157.01, 156.98, 156.85, 156.30, 143.31, 125.59, 119.64, 117.32, 115.00, 112.67, 111.19, 97.37, 95.62, 85.02, 78.58, 77.41, 77.03, 74.21, 72.50, 71.65, 70.76, 69.42, 69.37, 69.29, 69.25, 69.03, 68.98, 68.80, 66.53, 62.95, 55.18, 53.03, 51.75, 50.19, 41.67, 41.56, 38.63, 35.68, 31.82, 31.63, 29.47, 29.41, 29.36, 29.26, 29.11, 28.98, 28.62, 25.49, 22.33, 13.58.

HRMS: Calculated [M+2H]₂₊: 673.4230, found: 673.4226.

### Example 3. Preparation of GNeo-Containing Liposomes

A mixture (15 mg total) of DOPC (1,2-dioleoyl-sn-glycero-3-phosphocholine), DOPE (1,2-dioleoyl-sn-glycero-3-phosphoethanolamine) and cholesterol (73:11:16) was dissolved in chloroform to a final volume of 1 mL and evaporated in a round flask to form a thin lipid layer. Any solvent remaining was removed under high vacuum overnight. The resulting film was hydrated for 10 minutes at 40° C. with 1 mL of PBS containing stearyl-GNeo (0.36 mg, 0.9 mol %) and the cargo (any of the following: a water soluble cyanine dye prepared as reported,²⁹ 100 μM; Streptavidin-Cy3, 0.6 mg mL⁻¹; LysoSensor™ yellow/blue dextran, 1 mg mL⁻¹; fluorescein di-β-d-galactopyranoside, 200 μM; sulforhodamine, 20 mM). The mixture was sonicated for 30 seconds to completely detach the lipids, forming a fine suspension, and subjected to six freeze/thaw cycles using a dry-ice/acetone bath (1 minute) and a water bath at 40° C. (1.5 minutes). Finally the suspension was extruded 17 times through a polycarbonate membrane (pore size 100 nm) at room temperature. Extravesicular components were removed by gravitational gel filtration (Sephadex G-50 for small molecules or Sepharose 4B for dextran and streptavidin derivatives) eluting with PBS. The desired size distribution was verified by Dynamic Light Scattering analysis and lipid concentration was determined adapting the Stewart method.⁵⁰

Plain liposomes were prepared as described above without adding stearyl-GNeo.

### Example 4. Determination of Lipids in Liposomal Suspensions

After purification by size exclusion, lipid concentration was determined adapting the Stewart method.⁵⁰ Briefly, an aliquot of the liposomal suspension was diluted to an approximate lipid concentration of 0.25 mg/mL. To 2.5 mL of chloroform, 100 μL of the diluted liposomes were added followed by 2.5 mL of ammonium ferrothiocyanate (0.1 M). The biphasic system was vigorously vortexed for 20 seconds and further centrifuged for 1 minute. The optical density of the organic phase was measured at 480 nm against chloroform as a blank. The amount of lipids present was estimated by comparison to a calibration curve generated using liposomal suspensions with a known lipid content.

### Example 5. Cellular Uptake

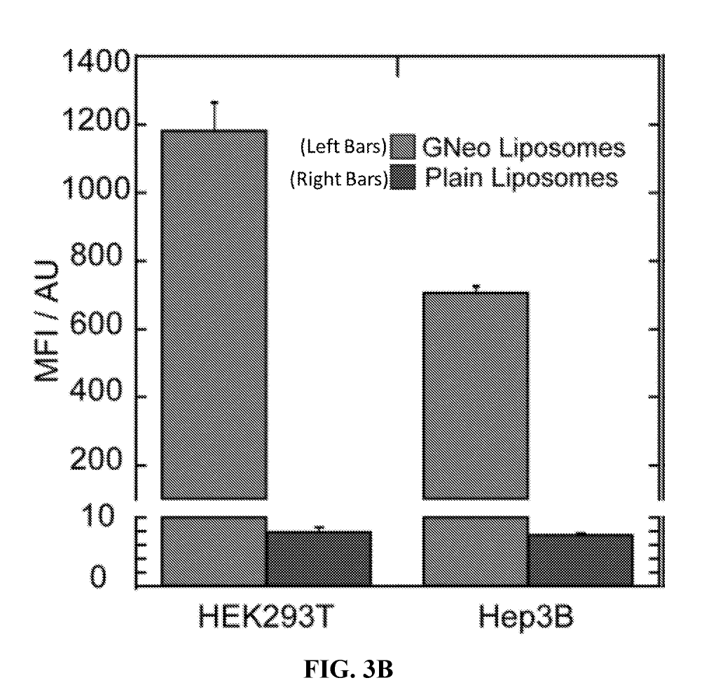

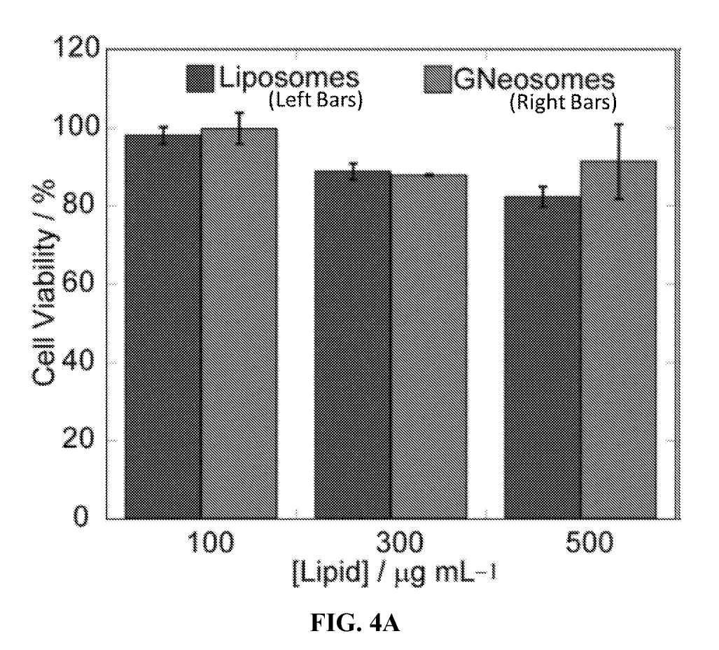

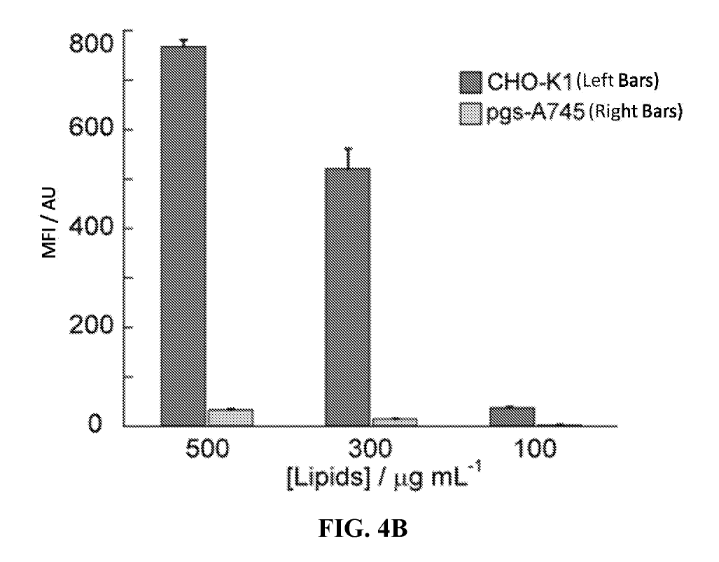

To compare and contrast the cellular uptake of plain and GNeo-containing liposomes, a water soluble cyanine dye²⁹ was packaged as an emissive small molecule. Uptake was evaluated in wild type CHO-K1 cells. The cells were incubated with liposomes at 37° C. for 1 hr, harvested and analyzed by flow cytometry. The mean fluorescence intensity (MFI) of the cells treated with decorated liposomes is remarkably higher compared to that arising from cells treated with plain liposomes, as shown in FIGS. 3A-3B.³⁰ The most effective formulation consisted of DOPC:DOPE:cholesterol:GNeo, 73:11:16:0.9. These vesicles, termed GNeosomes, which showed no cytotoxicity when incubated with CHO-K1 cells for 24 h at 0.1, 0.3 and 0.5 mg mL⁻¹ (see FIG. 4A), were selected for further investigations.

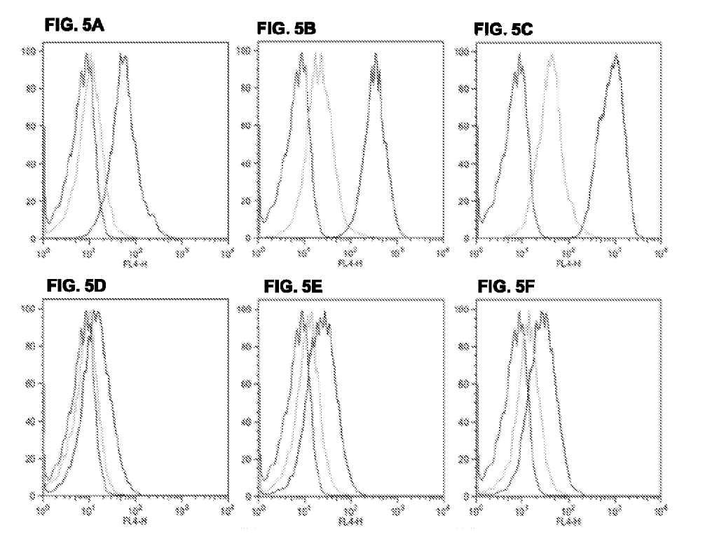

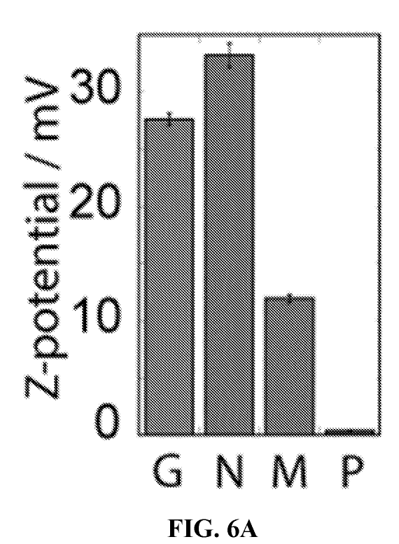

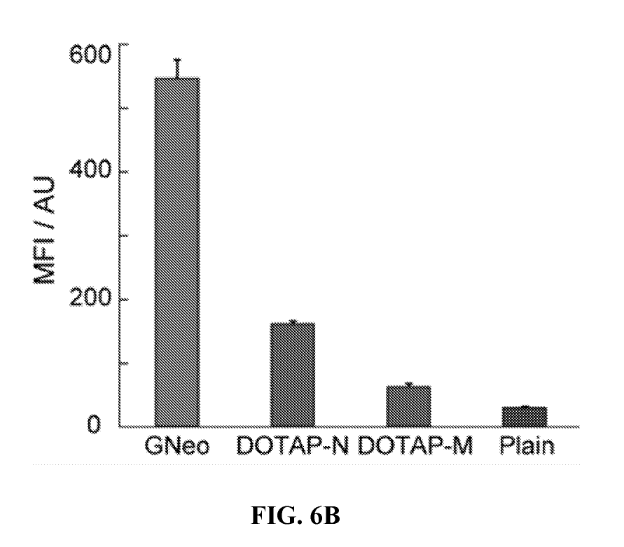

Cellular uptake of GNeosomes is dose dependent and highly selective for glycosaminoglycans (GAG), displaying extremely reduced cellular uptake in a mutant pgsA-745 cell line, which lacks heparan sulfate and chondroitin/dermatan sulfate as shown in FIG. 4B.³¹ Importantly, in addition to their low overall cellular uptake, plain liposomes display no selectivity for either cell line (see FIG. 5A-F). Further, to substantiate that this remarkable cellular uptake relies on the molecular nature of GNeo and not merely on augmenting the positive charge of the carrier surface, liposomes decorated with DOTAP (1,2-dioleoyl-3-trimethylammonium-propane), a cationic lipid, were evaluated. Lipid vesicles were prepared with 0.9 or 5.4 mol % DOTAP (DOTAP-M and DOTAP-N, respectively), to reflect an equimolar or equinormal ratio compared to GNeo. To evaluate the surface charge of these liposomes, their zeta potential was measured (see FIG. 6A and Table 1).

The presence of DOTAP in the hydration buffer raises the zeta potential of the formed liposomes from 0.4 to 12 and 33 mV (plain liposomes, DOTAP-M and DOTAP-N, respectively). While introducing GNeo to the lipid vesicles increases the zeta potential only to 27 mV, flow cytometry revealed that GNeosomes display a 8.7 and 3.4 fold increase in cellular uptake when compared to DOTAP-M and DOTAP-N, respectively (see FIG. 6B).²⁸ These observations unequivocally demonstrate that the superior enhancement in cellular uptake imparted by GNeo is neither simply due to charge effect only nor a common feature of cationic liposomes. It highlights the importance of the 3D structure of guanidinoneomycin and the presentation of its guanidinium groups and supports the competent delivery capabilities and inherent affinity and selectivity of GNeosomes for cell surface glycosaminoglycans.

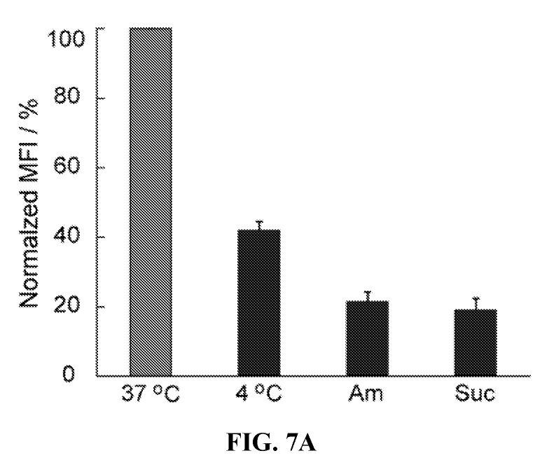

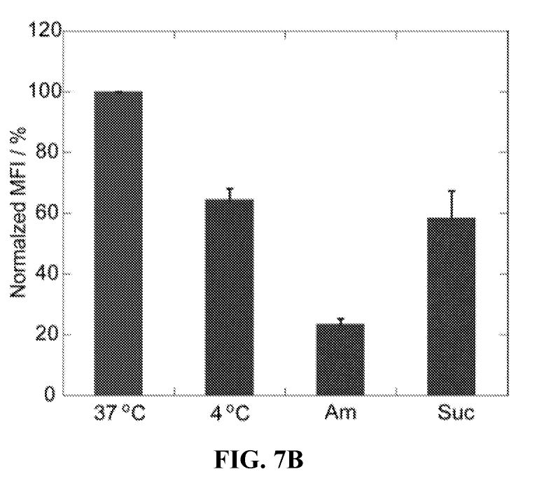

To better understand the internalization mechanism(s) of these previously unexplored lipid vesicles, the contribution of different endocytotic pathways was evaluated. Cellular uptake was therefore tested at low temperature (assessing the contribution of energy-dependent processes) and in cells pretreated with sucrose (impeding clathrin-mediated endocytosis) or with amiloride (hindering macropinocytosis). Interestingly, the internalization of GNeosomes and plain liposomes at low temperatures decreases to 40% and 60%, respectively, as shown in FIGS. 7A-7B. Notably, in cells pretreated with sucrose, the internalization of plain liposomes was also lowered to 60% while the entry of GNeosomes was reduced to 20% as shown in FIGS. 7A-7B. Furthermore, unlike the effect seen for GNeo-protein conjugates,¹⁶ pre-treating the cells with amiloride reduces the internalization of GNeo-based nanocarriers by 80%. This reduction in uptake was also observed for plain liposomes as shown in FIGS. 7A-7B. Taken together, these results suggest that energy-dependent pathways are involved in the internalization of GNeosomes to a higher extent compared to plain liposomes. Background energy-independent internalization does take place, however, as uptake is also seen at low temperatures, as has been reported for certain liposomes and other carriers.³²⁻³⁵

### Example 6. Intracellular Localization

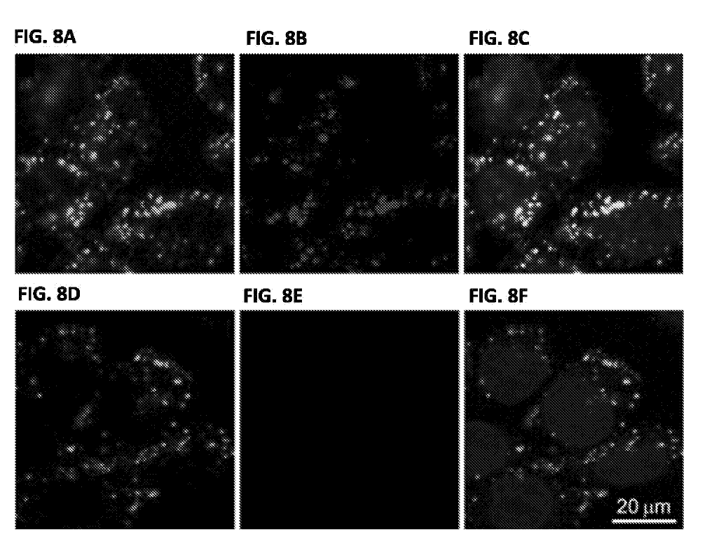

To elucidate the intracellular fate of these lipid vesicles, live cells were imaged by Confocal Laser Scanning Microscopy (CLSM) after one hour incubation with carriers loaded with a water soluble Cy5 dye and further treatment with the lysosomal marker LysoTracker Green DND-26 and the nuclear stain Hoechst 33342. Overlaying the images from the green and far red (pseudocolored in red) channels reveals a high degree of colocalization for GNeosomes and LysoTracker stained compartments while practically no colocalization is observed for plain liposomes, resulting in Pearson's correlation of 0.82 and 0.11, respectively, as shown in FIGS. 8A-8F. This observation suggests that decorating the liposomes with GNeo not only drastically enhances their cellular uptake but, most importantly, selectively directs them to the target organelle.

### Example 7. Delivery of Biomacromolecules

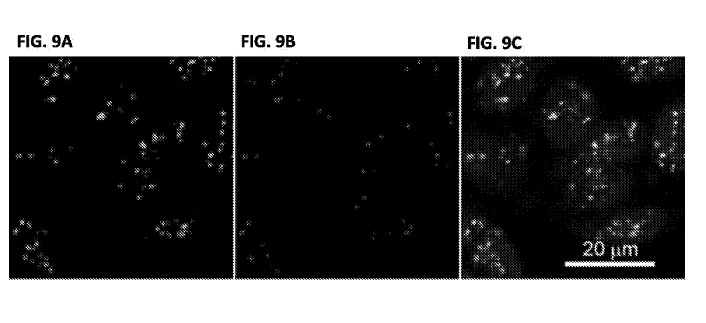

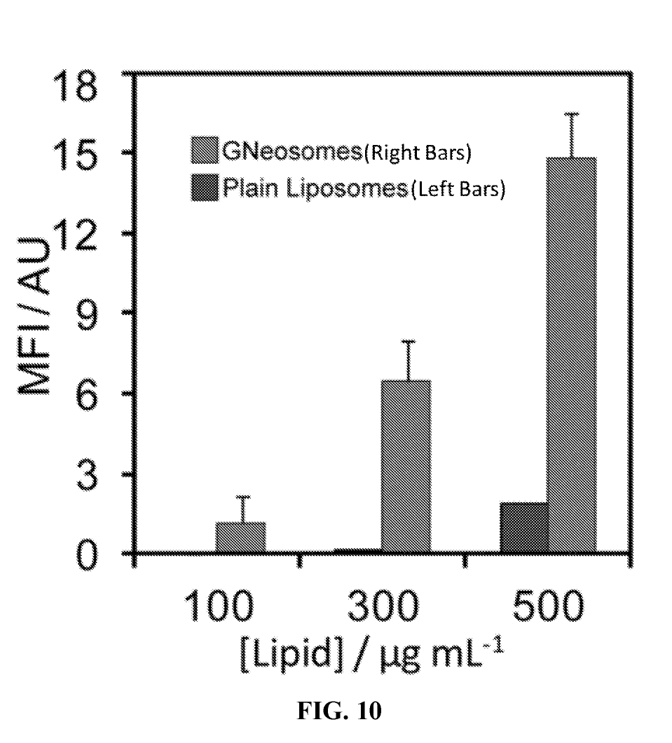

To investigate the cellular delivery of high molecular weight proteins, GNeosomes were loaded with a fluorescently labeled streptavidin (ST-Cy3; 60 KDa). Cells were incubated with GNeosomes for one hour and further treated with the lysosomal marker LysoTracker Green DND-26 and the nuclear stain Hoechst 33342. Overlaying the images from the green and red channels reveals a high degree of colocalization for GNeosomes and LysoTracker stained organelles, as shown in FIGS. 9A-9C. Moreover, flow cytometry analysis comparing the cellular delivery of ST-Cy3 loaded either in plain liposomes or in GNeosomes shows a high increase in the MFI for the cells treated with the latter, as shown in FIG. 10. Importantly, live cell imaging together with flow cytometry analysis unambiguously demonstrate the ability of GNeosomes to efficiently deliver these biomacromolecules to the lysosomes.

### Example 8. Lysosomal Release of Cargo

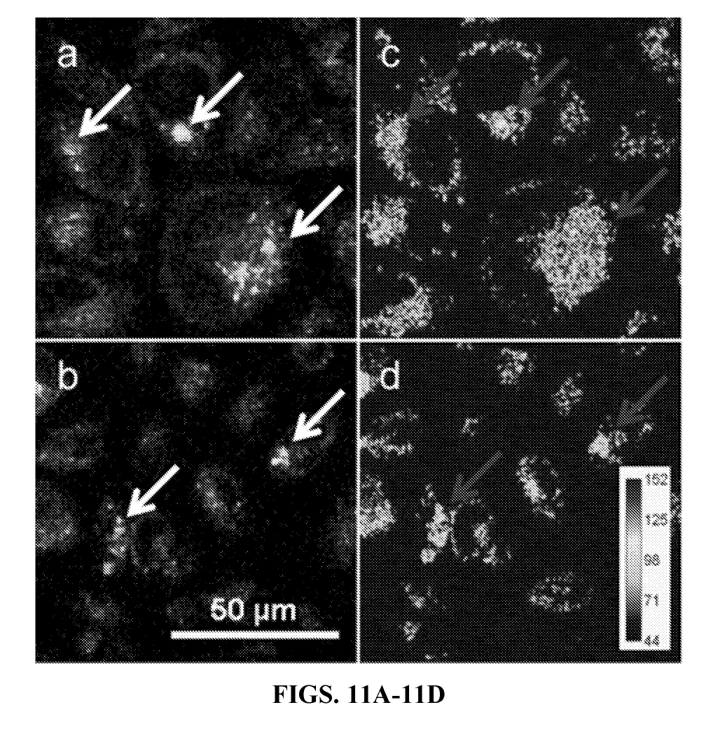

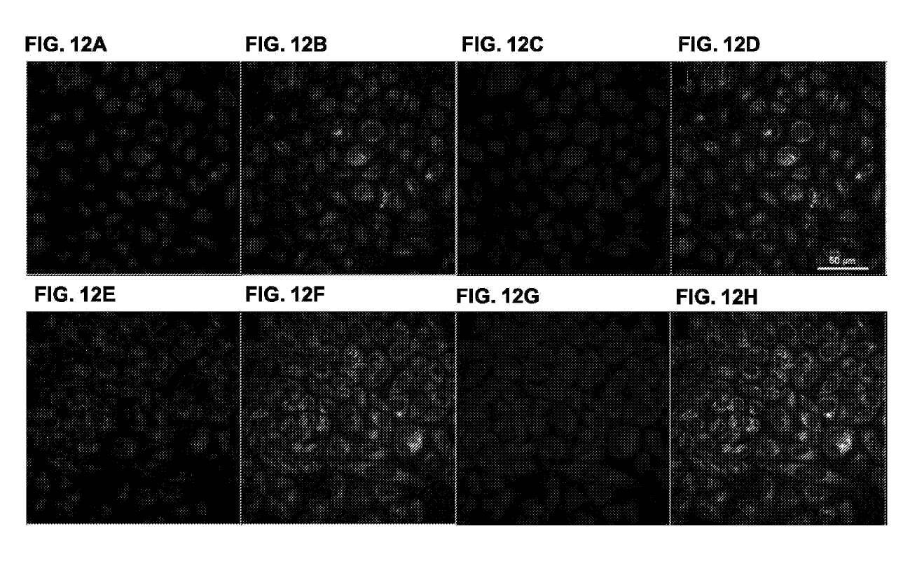

While cellular internalization is one step, intracellular trafficking and release are fundamental for cellular investigations or therapeutic applications. To confirm that the cargo loaded in GNeosomes is released in the lysosomes, LysoSensor™ Dextran Blue/Yellow (MW=10.000) was used. The readout of this intracellular pH indicator is based on a ratiometric analysis between the fluorescent intensity of “green” and “blue” emission bands, which increases as pH decreases.³⁶ CHO-K1 cells were incubated either with GNeosomes loaded with LysoSensor™ or with non-encapsulated LysoSensor™ and live cells were imaged by CLSM using LysoTracker Deep Red to mark the lysosomes. Overlaying the LysoTracker signal with either the “blue” or “green” channels clearly shows higher lysosomal colocalization for GNeosomes compared to the non-encapsulated LysoSensor™, as shown in FIGS. 11A-B and FIGS. 12A-12H. Furthermore, the areas in which colocalization is more evident (see e.g., FIGS. 11A-11B, white arrows), show the highest intensity in the ratiometric images, as shown in FIGS. 11C-11D, gray arrows). This correlation strongly suggests that the cargo, originally encapsulated in GNeosomes at pH 7.4, was released into a more acidic environment resembling that surrounding the non-encapsulated LysoSensor™, and is thus found free in the lysosomes.

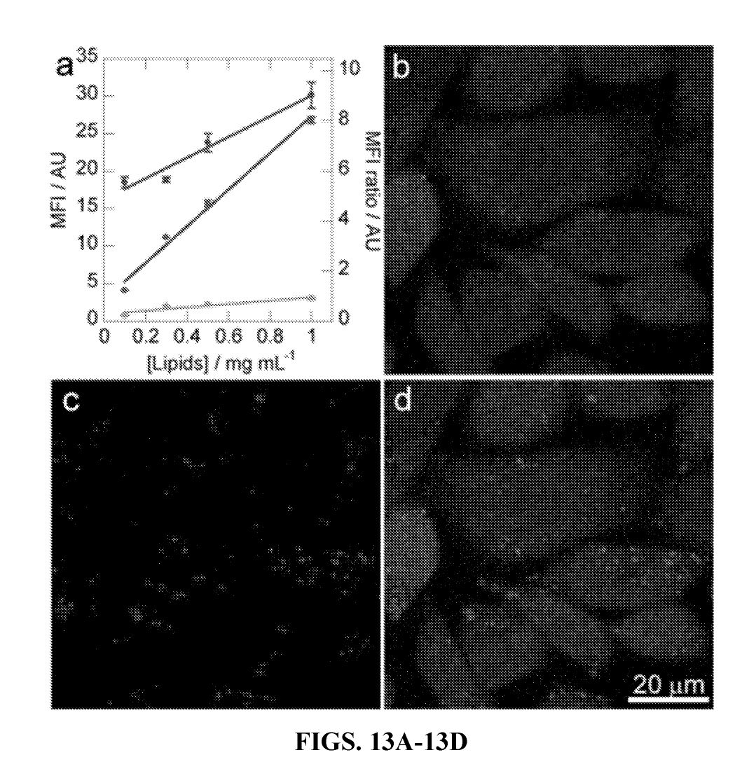

To further quantify the increase in lysosomotropism of GNeosomes compared to plain liposomes, these lipid vesicles were loaded with fluorescein di-β-D-galactopyranoside (FDG), a fluorogenic substrate for the intralysosomal enzyme β-galactosidase (β-Gal). Upon hydrolysis by β-Gal in the lysosome, the released fluorescein can be quantified by FACS and visualized by CLSM. Incubating wild type cells with either GNeo-decorated or plain liposomes and further quantification of the lysosomal delivery demonstrates that GNeosomes are remarkably more lysosomotropic than plain liposomes, as the ratio between the MFI observed for GNeosomes and that for plain liposomes shows up to nine fold enhancement under these experimental conditions, as shown in FIG. 13A and below in Table 2. CHO-K1 cells were incubated for one hour with GNeosomes or plain liposomes at the indicated concentrations. The background signal from untreated cells was subtracted and the ratio between the signals from GNeosomes and plain liposomes was calculated.

TABLE 2
^{a}Flow cytometry of lysosomal targeting by
liposomes loaded with FDG.

| | | | |
|---|---|---|---|
| | | | GNeosomes/ |
| [Lipid] | GNeosomes | Liposomes | Liposomes |

| mg/mL | MFI | SD | MFI | SD | MFI | SD |
|---|---|---|---|---|---|---|
| 0.1 | 4.12 | 0.03 | 0.74 | 0.03 | 5.57 | 0.22 |
| 0.3 | 11.7 | 0.56 | 1.975 | 0.035 | 5.92 | 0.10 |
| 0.5 | 15.72 | 0.45 | 2.205 | 0.095 | 7.14 | 0.37 |
| 1 | 26.77 | 0.4 | 2.975 | 0.165 | 9.03 | 0.52 |

^{a}MFI: Mean Fluorescence Intensity. SD: Standard Deviation.

The dose-dependent increase in the signal arising from the enzymatic activity of β-Gal implies that the lysosomal presence of GNeosomes, intact or degraded, does not inhibit the enzyme. Interestingly, live cell imaging of wild-type CHO-K1 treated with GNeosomes encapsulating FDG reveals diffuse green fluorescence, consistent with fluorescein's lysosomal escape as reported by Straubinger et al. and shown in FIG. 13B.³⁷ Furthermore, as observed in FIGS. 8A-8F, 9A-9C, and 11A-11D, the punctated appearance of the lysosomes is not disrupted, suggesting that their integrity is maintained, as shown in FIGS. 13C-13D. These observations are in agreement with the high degree of lysosomal colocalization displayed by GNeosomes, reinforcing the notion of GNeo-enhanced lysosomotropism.

### Example 9. Preparation of Additional Liposomal GNeosome Derivatives

FIG. 14 compares three methodologies for preparing lipidated GNeosome derivatives. Pre-inserted: Lipidated GNeo is premixed with other lipids for liposomes formation (labeled as “A” in FIG. 14). Post-inserted: “Plain” liposomes are first made and then equilibrated with lipidated GNeo (0.9 and 1.8% are shown and labeled as “B” and “C”, respectively, in FIG. 14). Post-modification: “Plain” liposomes, containing PE are reacted with GNeo-NHS for surface modification (labeled as “D” in FIG. 14).

Liposomes were prepared with Cy5. Wild-type CHO-K1 cells were then incubated with 0.3 mg/mL plain liposomes or GNeosomes for 1 h at 37° C. Mean fluorescence intensity (MFI) was measured by flow cytometry.

### Example 10. Cargo Delivery into MPS Cells

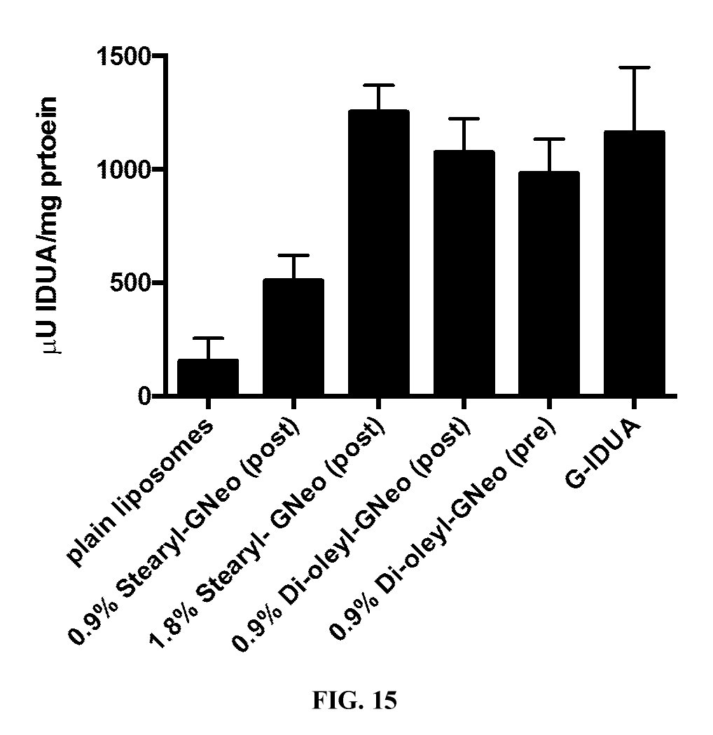

Liposomes were prepared with α-L-Iduronidase (IDUA, a lysosomal enzyme). MPS I fibroblasts were incubated with 0.5 mg/mL plain liposomes or GNeosomes for 1 h at 37° C. The cells were washed, trypsin treated, and lysed then assayed for IDUA activity by measuring the conversion of 4-methylumbelliferyl α-L-iduronide into the fluorochrome 4-methylumbelliferone (4-MU). Cells were treated with the same concentration of GNeo-IDUA for comparison. FIG. 15 shows results of delivering IDUA into MPS cells taken from patients using “plain” liposomes and four lipidated GNeosome derivatives. The terms “post” and “pre” in FIG. 15 refer to the preparation methodology as described above in Example 9.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

### REFERENCES

(1) Luzio, J. P.; Pryor, P. R.; Bright, N. A. Lysosomes: Fusion and Function. Nat. Rev. Mol. Cell. Bio. 2007, 8, 622-632.
(2) Winchester, B.; Vellodi, A.; Young, E. The Molecular Basis of Lysosomal Storage Diseases and their Treatment. Biochem. Soc. T. 2000, 28, 150-154.
(3) Sidransky, E.; Lopez, G. The Link Between the GBA Gene and Parkinsonism. Lancet Neurol. 2012, 11, 986-998.
(4) Westbroek, W.; A. Gustafson, M.; Sidransky, E. Exploring the Link Between Glucocerebrosidase Mutations and Parkinsonism. Trends Mol. Med. 2011, 17, 485-493.
(5) Appelqvist, H.; Wäster, P.; Kågedal, K.; Öllinger, K. The Lysosome: From Waste Bag to Potential Therapeutic Target. J. Mol. Cell Biol. 2013, 5, 214-226.
(6) Rin Jean, S.; Tulumello, D. V.; Wisnovsky, S. P.; Lei, E. K.; Pereira, M. P.; Kelley, S. O. Molecular Vehicles for Mitochondrial Chemical Biology and Drug Delivery. ACS Chem. Biol. 2014, 9, 323-333.
(7) Torchilin, V. P. Recent Approaches to Intracellular Delivery of Drugs and DNA and Organelle Targeting. Annu. Rev. Biomed. Eng. 2006, 8, 343-375.
(8) Giannotti, M. I.; Esteban, O.; Oliva, M.; Garcia-Parajo, M. F.; Sanz, F. pH-Responsive Polysaccharide-Based Polyelectrolyte Complexes As Nanocarriers for Lysosomal Delivery of Therapeutic Proteins. Biomacromolecules 2011, 12, 2524-2533.
(9) Maniganda, S.; Sankar, V.; Nair, J. B.; Raghu, K. G.; Maiti, K. K. A Lysosome-targeted Drug Delivery System Based on Sorbitol Backbone Towards Efficient Cancer Therapy. Org. Biomol. Chem. 2014, 12, 6564-6569.
(10) Oh, N. M.; Oh, K. T.; Youn, Y. S.; Lee, D.-K.; Cha, K.-H.; Lee, D. H.; Lee, E. S. Poly(L-aspartic acid) Nanogels for Lysosome-selective Antitumor Drug Delivery. Colloids Surf B 2013, 101, 298-306.
(11) Zhu, S.; Lansakara-P, D. S. P.; Li, X.; Cui, Z. Lysosomal Delivery of a Lipophilic Gemcitabine Prodrug Using Novel Acid-Sensitive Micelles Improved Its Antitumor Activity. Bioconjugate Chem. 2012, 23, 966-980.
(12) Nair, J. B.; Mohapatra, S.; Ghosh, S.; Maiti, K. K. Novel Lysosome Targeted Molecular Transporter Built on a Guanidinium-poly-(propylene imine) Hybrid Dendron for Efficient Delivery of Doxorubicin into Cancer Cells. Chem Commun 2015, 51, 2403-2406.
(13) Meerovich, I.; Koshkaryev, A.; Thekkedath, R.; Torchilin, V. P. Screening and Optimization of Ligand Conjugates for Lysosomal Targeting. Bioconjugate Chem. 2011, 22, 2271-2282.
(14) Baker, T. J.; Luedtke, N. W.; Tor, Y.; Goodman, M. Synthesis and anti-HIV activity of guanidinoglycosides. J. Org. Chem. 2000, 65, 9054-9058.
(15) Luedtke, N. W.; Carmichael, P.; Tor, Y. Cellular Uptake of Aminoglycosides, Guanidinoglycosides, and poly-Arginine. J. Am. Chem. Soc. 2003, 125, 12374-12375.
(16) Elson-Schwab, L.; Garner, O. B.; Schuksz, M.; Crawford, B. E.; Esko, J. D.; Tor, Y. Guanidinylated Neomycin Delivers Large, Bioactive Cargo into Cells Through a Heparan Sulfate-Dependent Pathway. J. Biol. Chem. 2007, 282, 13585-13591.
(17) Bishop, J. R.; Schuksz, M.; Esko, J. D. Heparan Sulphate Proteoglycans Fine-Tune Mammalian Physiology. Nature 2007, 446, 1030-1037.
(18) Xu, D.; Esko, J. D. Demystifying Heparan Sulfate-Protein Interactions. Annu. Rev. Biochem. 2014, 83, 129-157.
(19) Sarrazin, S.; Wilson, B.; Sly, W. S.; Tor, Y.; Esko, J. D. Guanidinylated Neomycin Mediates Heparan Sulfate-Dependent Transport of Active Enzymes to Lysosomes. Mol. Ther. 2010, 18, 1268-1274
(20) Dix, A. V.; Fischer, L.; Sarrazin, S.; Redgate, C. P.; Esko, J. D.; Tor, Y. Cooperative, Heparan Sulfate-Dependent Cellular Uptake of Dimeric Guanidinoglycosides. ChemBioChem 2010, 11, 2302-2310.
(21) Inoue, M.; Tong, W.; Esko, J. D.; Tor, Y. Aggregation-Mediated Macromolecular Uptake by a Molecular Transporter. ACS Chem. Biol. 2013, 8, 1383-1388.
(22) Natarajan, A.; Du, W.; Xiong, C. Y.; DeNardo, G. L.; DeNardo, S. J.; Gervay-Hague, J. Construction of di-scFv through a Trivalent Alkyne-Azide 1,3-Dipolar Cycloaddition. Chem. Commun. 2007, 7, 695-697.
(23) Related derivatives were reported. See references 24-27.
(24) Sainlos, M.; Belmont, P.; Vigneron, J.-P.; Lehn, P.; Lehn, J.-M. Aminoglycoside-Derived Cationic Lipids for Gene Transfection: Synthesis of Kanamycin A Derivatives. Eur. J. Org. Chem. 2003, 15, 2764-2774.
(25) Sainlos, M.; Hauchecorne, M.; Oudrhiri, N.; Zertal-Zidani, S.; Aissaoui, A.; Vigneron, J.-P.; Lehn, J.-M.; Lehn, P. Kanamycin A-Derived Cationic Lipids as Vectors for Gene Transfection. ChemBioChem 2005, 6, 1023-1033.
(26) Mével, M.; Sainlos, M.; Chatin, B.; Oudrhiri, N.; Hauchecorne, M.; Lambert, O.; Vigneron, J.-P.; Lehn, P.; Pitard, B.; Lehn, J.-M. Paromomycin and Neomycin B Derived Cationic Lipids: Synthesis and Transfection Studies. J. Control. Release 2012, 158, 461-469.
(27) Bera, S.; Dhondikubeer, R.; Findlay, B.; Zhanel, G. G.; Schweizer, F. Synthesis and Antibacterial Activities of Amphiphilic Neomycin B-based Bilipid Conjugates and Fluorinated Neomycin B-based Lipids. Molecules 2012, 17, 9129-9141.
(28) Encapsulation efficiency remains unaffected by the addition of 4 (Figure S2).
(29) Kojima, R.; Takakura, H.; Ozawa, T.; Tada, Y.; Nagano, T.; Urano, Y. Rational Design and Development of Near-Infrared-Emitting Firefly Luciferins Available in vivo. Angew. Chem. Int. Ed. 2013, 52, 1175-1179.
(30) Our observations are not limited to CHO cells. Substantially enhanced uptake was also seen in HEK293T and Hep3B cell lines. See Figure S3.
(31) Esko, J. D.; Stewart, T. E.; Taylor, W. H. Animal-Cell Mutants Defective in Glycosaminoglycan Biosynthesis. Proc. Natl. Acad. Sci. USA 1985, 82, 3197-3201.
(32) Gasparini, G.; Bang, E.-K.; Molinard, G.; Tulumello, D. V.; Ward, S.; Kelley, S. O.; Roux, A.; Sakai, N.; Matile, S. Cellular Uptake of Substrate-Initiated Cell-Penetrating Poly(disulfide)s. J. Am. Chem. Soc. 2014, 136, 6069-6074.
(33) Iwasa, A.; Akita, H.; Khalil, I.; Kogure, K.; Futaki, S.; Harashima, H. Cellular Uptake and Subsequent Intracellular Trafficking of R8-Liposomes Introduced at Low Temperature. BBA-Biomembranes 2006, 1758, 713-720.
(34) Mitchell, D. J.; Steinman, L.; Kim, D. T.; Fathman, C. G.; Rothbard, J. B. Polyarginine Enters Cells More Efficiently than Other Polycationic Homopolymers. J. Pept. Res. 2000, 56, 318-325.
(35) Torchilin, V. P.; Rammohan, R.; Weissig, V.; Levchenko, T. S. TAT Peptide on the Surface of Liposomes Affords their Efficient Intracellular Delivery Even at Low Temperature and in the Presence of Metabolic Inhibitors. Proc. Natl. Acad. Sci. USA 2001, 98, 8786-8791.
(36) Diwu, Z. J.; Chen, C. S.; Zhang, C. L.; Klaubert, D. H.; Haugland, R. P. A Novel Acidotropic pH Indicator and its Potential Application in Labeling Acidic Organelles of Live Cells. Chem. Biol. 1999, 6, 411-418.
(37) Straubinger, R. M.; Hong, K.; Friend, D. S.; Papahadjopoulos, D. Endocytosis of Liposomes and Intracellular Fate of Encapsulated Molecules—Encounter with a Low pH Compartment after Internalization in Coated Vesicles. Cell 1983, 32, 1069-1079.
(38) Chang, H. I.; Yeh, M. K. Clinical Development of Liposome-Based Drugs: Formulation, Characterization, and Therapeutic Efficacy. Int. J. Nanomed. 2012, 7, 49-60.
(39) Slingerland, M.; Guchelaar, H. J.; Gelderblom, H. Liposomal Drug Formulations in Cancer Therapy: 15 Years Along the Road. Drug Discov. Today 2012, 17, 160-166.
(40) Torchilin, V. P. Cell Penetrating Peptide-Modified Pharmaceutical Nanocarriers for Intracellular Drug and Gene Delivery. I Pept. Sci. 2008, 90, 604-610.
(41) Cryan, S.-A.; Devocelle, M.; Moran, P. J.; Hickey, A. J.; Kelly, J. G. Increased Intracellular Targeting to Airway Cells Using Octaarginine-Coated Liposomes: In Vitro Assessment of Their Suitability for Inhalation. Mol. Pharm. 2006, 3, 104-112.
(42) Console, S.; Marty, C.; Garcia-Echeverria, C.; Schwendener, R.; Ballmer-Hofer K. Antennapedia and HIV Transactivator of Transcription (TAT) “Protein Transduction Domains” Promote Endocytosis of High Molecular Weight Cargo upon Binding to Cell Surface Glycosaminoglycans. J. Biol. Chem. 2003, 278, 35109-35114.
(43) Marty, C.; Meylana, C.; Schottb, H.; Ballmer-Hofer, K.; Schwendener, R. A. Enhanced heparan sulfate proteoglycan-mediated uptake of cell-penetrating peptide-modified liposomes. Cell. Mol. Life Sci. 2004, 61, 1785-1794.
(44) Biswas, S.; Dodwadkar, N. S.; Deshpande, P. P.; Torchilin, V. P. Liposomes Loaded with Paclitaxel and Modified with Novel Triphenylphosphonium-PEG-PE Conjugate Possess Low Toxicity, Target Mitochondria and Demonstrate Enhanced Antitumor Effects In Vitro and In Vivo. J. Control. Release 2012, 159, 393-402.
(45) Koshkaryev, A.; Thekkedath, R.; Pagano, C.; Meerovich, I.; Torchilin, V. P. Targeting of Lysosomes by Liposomes Modified with Octadecyl-Rhodamine B. J. Drug Target 2011, 19, 606-614.
(46) Pollock, S.; Antrobus, R.; Newton, L.; Kampa, B.; Rossa, J.; Latham, S.; Nichita, N. B.; Dwek, R. A.; Zitzmann, N. Uptake and Trafficking of Liposomes to the Endoplasmic Reticulum. FASEB J. 2010, 24, 1866-1878.
(47) Koshkaryev, A.; Piroyan, A.; Torchilin, V. P. Increased Apoptosis in Cancer Cells In Vitro and In Vivo by Ceramides in Transferrin-Modified Liposomes. Cancer Biol. Ther. 2012, 13, 50-59.
(48) Thekkedath, R.; Koshkaryev, A.; Torchilin, V. P. Lysosome-Targeted Octadecyl-Rhodamine B-Liposomes Enhance Lysosomal Accumulation of Glucocerebrosidase in Gaucher's Cells In Vitro. Nanomedicine 2013, 8, 1055-1065.
(49) Muro, S.; Schuchman, E. H.; Muzykantov, V. R. Lysosomal Enzyme Delivery by ICAM-1-targeted Nanocarriers Bypassing Glycosylation- and Clathrin-dependent Endocytosis. Mol. Ther. 2006, 13, 135-141.
(50) Stewart, J. C. M. Colorimetric Determination of Phospholipids with Ammonium Ferrothiocyanate. Anal. Biochem. 1980, 104, 10-14.
(51) Bai, X. M.; Wei, G.; Sinha, A.; Esko, J. D. Chinese Hamster Ovary Cell Mutants Defective in Glycosaminoglycan Assembly and Glucuronosyltransferase I. J. Biol. Chem. 1999, 274, 13017-13024.

What is claimed is:

1. A compound of Formula I:
A-B-C I
or a pharmaceutically acceptable salt thereof, wherein:
A is a guanidinylated neomycin derivative;
B is a linker group; and
C is a phospholipid, a fatty acid, or a fatty acid group.

2. The compound of Formula I, wherein A is a guanidinylated neomycin derivative of the following formula: 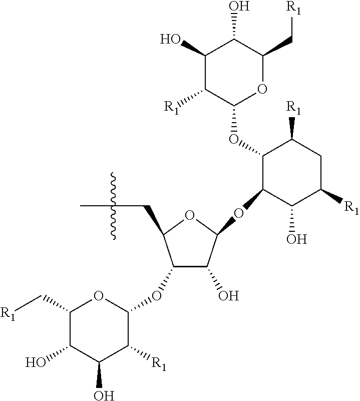
wherein:
R₁ is a guanidine or guanidinium group; and [symbol]
indicates the bond between A and B of Formula I.

3. The compound of claim 2, wherein the guanidine group is an N-protected guanidine.

4. The compound of claim 3, wherein the N-protected guanidine group is of the following formula: 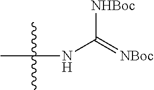
wherein Boc is tert-butoxycarbonyl.

5. The compound of claim 2, wherein the guanidinium group is of the following formula: 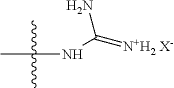
wherein X⁻ is an anion.

6. The compound of claim 2, wherein the guanidinium group is of the following formula:

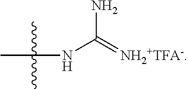

7. The compound of any one of claims 1 to 6, wherein B is a linker group comprising a linker selected from the group consisting of one or more alkylene groups, one or more amide groups, one or more alkyleneoxy groups, one or more heteroaryl groups, one or more amine groups, or any combination thereof.

8. The compound of any one of claims 1 to 6, wherein B is a linker group comprising one or more C₁₋₁₀ alkylene groups, one or more —(OCH₂CH₂)— or —(OCH₂)— groups, one amide group, one —NH— group, and one 5-6 membered heteroaryl group.

9. The compound of any one of claims 1 to 6, wherein B is a linker group selected from the group of the following formulae: 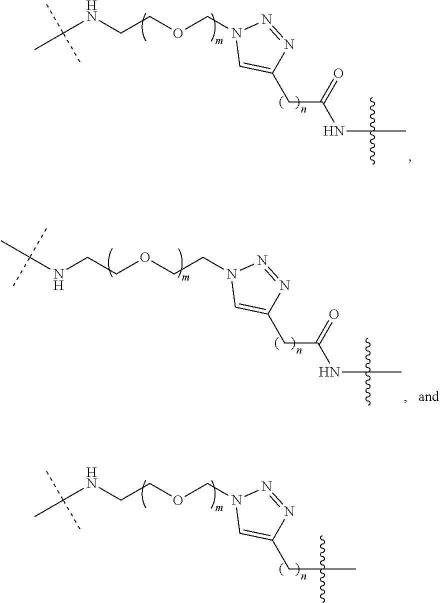
wherein:
m is an integer from 1 to 10;
n is an integer from 0 to 10; [symbol]
indicates the bond between A and B of Formula I; and [symbol]
indicates the bond between B and C of Formula I.

10. The compound of claim 9, wherein m is an integer from 1 to 5.

11. The compound of claim 9, wherein m is 3.

12. The compound of any one of claims 9 to 11, wherein n is an integer from 0 to 5.

13. The compound of any one of claims 9 to 11, wherein n is 0 or 3.

14. The compound of any one of claims 1 to 13, wherein C is a phospholipid.

15. The compound of any one of claims 1 to 13, wherein C is a phospholipid comprising one or more choline groups, one or more glycerophosphoric acid groups, and one or more fatty acid groups.

16. The compound of any one of claims 1 to 13, wherein C is a phospholipid comprising one or more ethanolamine groups, one or more glycerophosphoric acid groups, and one or more fatty acid groups.

17. The compound of any one of claims 1 to 13, wherein C is a phosphatidylcholine.

18. The compound of any one of claims 1 to 13, wherein C is a phosphatidylcholine selected from the group consisting of POPC (1-palmitoyl-2-oleoyl-sn-glycero-3-phosphocholine), DOPC (1,2-dioleoyl-sn-glycero-3-phosphocholine), DOPE (1,2-dioleoyl-sn-glycero-3-phosphoethanolamine), or any combination thereof.

19. The compound of any one of claims 1 to 13, wherein C is a phospholipid, fatty acid or fatty acid group selected from the following formulae: 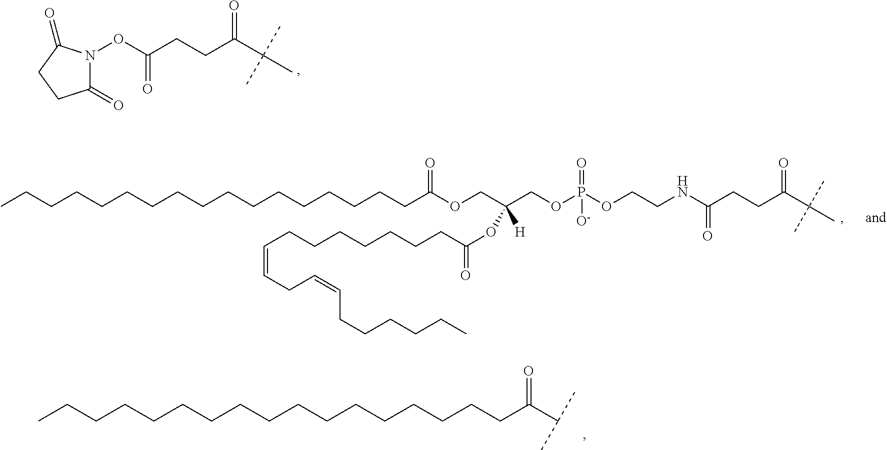
wherein: [symbol]
indicates the bond between B and C of Formula I.

20. The compound of claim 1, wherein the compound is selected from the group consisting of: 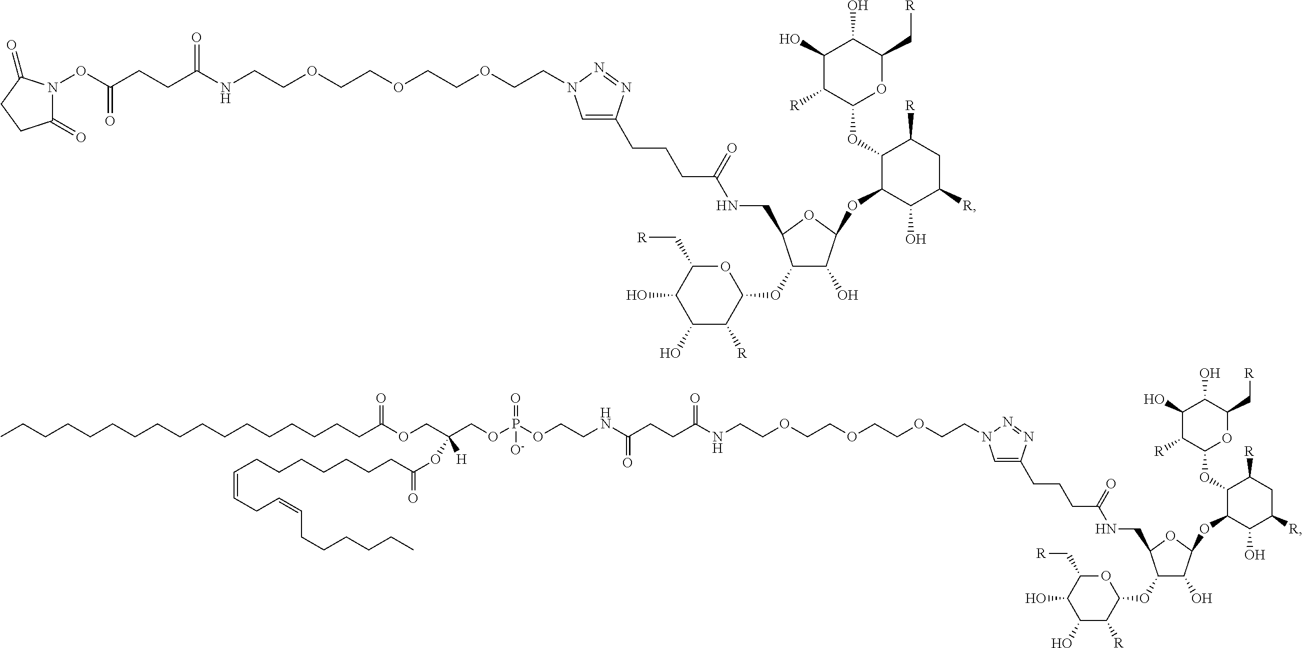 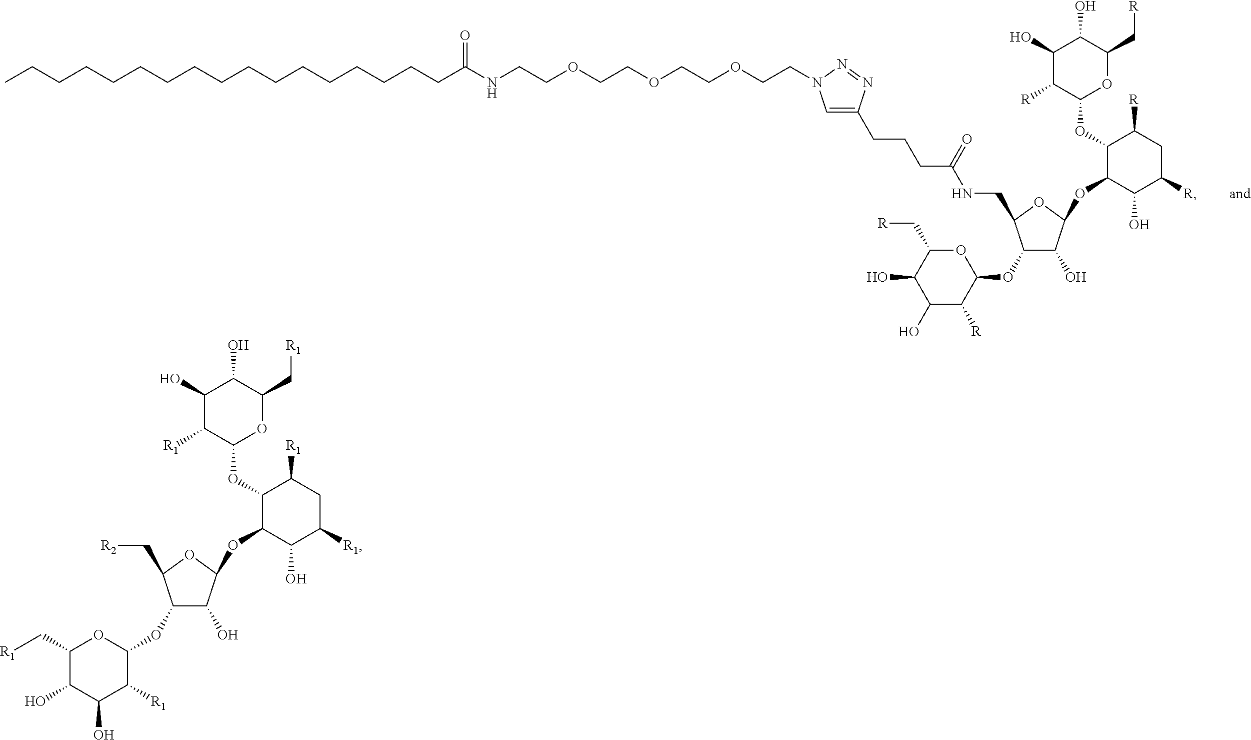
wherein:
each R is 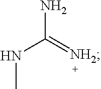
each R₁ is 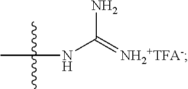
and
each R2 is

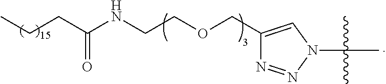

21. A conjugate, comprising a compound of any one of claims 1 to 21 and a liposome.

22. The conjugate of claim 21, wherein the liposome comprises a group selected from the group consisting of a POPC group (1-palmitoyl-2-oleoyl-sn-glycero-3-phosphocholine), a DOPC group (1,2-dioleoyl-sn-glycero-3-phosphocholine), DOPE group (1,2-dioleoyl-sn-glycero-3-phosphoethanolamine), and a cholesterol group, or any combination thereof.

23. The conjugate of claim 21 or 22, which is selected from the group consisting of POPC:Stearyl-GNeo, DOPC:Stearyl-GNeo, DOPC:DOPE:Stearyl-GNeo, and DOPC:DOPE:Cholesterol:Stearyl-GNeo.

24. The conjugate of claim 23, wherein the ratio of POPC:Stearyl-GNeo is about 100:1.

25. The conjugate of claim 23, wherein the ratio of DOPC:Stearyl-GNeo is about 100:0.9.

26. The conjugate of claim 23, wherein the ratio of DOPC:DOPE:Stearyl-GNeo it about 85:15:0.9.

27. The conjugate of claim 23, wherein ratio of DOPC:DOPE:Cholesterol:Stearyl-GNeo is about 73:11:16:0.9.

28. The conjugate of any one of claims 21 to 27, further comprising a therapeutic agent.

29. A method for treating a Lysosomal Storage Disorder in a patient in need thereof, comprising administering to the patient a conjugate of any one of claims 21 to 28.

30. A method for treating a central nervous system (CNS) disorder in a patient in need thereof, comprising administering to the patient a conjugate of any one of claims 21 to 28.

31. A method for treating a neurological disorder in a patient in need thereof, comprising administering to the patient a conjugate of any one of claims 21 to 28.
